(19) United States
(12) Patent Application Publication
(10) Pub. No.: US 2026/0049280 A1
(43) Pub. Date: Feb. 19, 2026

(54) CELLS FOR THERAPY

(71) Applicant: Resolution Therapeutics Limited, London (GB)

(72) Inventors: Kayleigh THIRLWELL, London (GB); Lara CAMPANA, London (GB); Rafael Alejandro ARMESILLA DIAZ, London (GB)

(73) Assignee: Resolution Therapeutics Limited, London (GB)

(21) Appl. No.: 19/104,682

(22) PCT Filed: Aug. 18, 2023
(86) PCT No.: PCT/EP2023/072801

§ 371 Date: Feb. 18, 2025

(30) Foreign Application Priority Data
Aug. 19, 2022 (GB) 2212144.6

(51) Int. Cl. C12N 5/0786; A61K 35/15

(52) U.S. Cl. CPC C12N 5/0645; A61K 35/15; C12N 2501/22; C12N 2501/2303; C12N 2501/231; C12N 2501/727; C12N 2506/45; C12N 2510/00

(57) ABSTRACT

The present invention relates to methods of producing hypoimmunogenic functional macrophages, and cell compositions comprising the same. The hypoimmunogenic functional macrophages may be polarised or unpolarised. Such cells may be useful in the treatment of diseases, e.g. inflammatory organ damage. The therapy that may be achieved by hypoimmunogenic functional macrophages is universal, and the cells do not require matching from donor to recipient. This broadens the application of cell-based therapies, in particular in patients with chronic liver fibrosis.

### FIELD OF THE INVENTION

The present invention relates to a polarised hypoimmunogenic stem cell derived macrophage suitable for use in treatment of an inflammatory condition in a subject e.g. inflammatory organ damage. The present invention further relates to populations of, compositions comprising, and methods using, such macrophages. The therapy that may be achieved is universal, and the cells do not require matching from donor to recipient. This broadens the application of cell-based therapies, in particular in patients with chronic liver fibrosis.

### BACKGROUND

Fibrosis is the final common pathway of chronic disease of various aetiologies, including toxic damage, viral infections, metabolic and genetic diseases, and autoimmune diseases. Acute, self-limiting fibrosis has likely evolved as a reversible and protective response to injury. The balance between self-limited and excessive fibrosis is finely regulated by multiple pathways and systems, and essentially dependent on the duration and repetition of the injury. A paradigm for the biology of fibrosis generation and remodelling is offered by the liver. End-stage, chronic liver fibrosis also known as cirrhosis, is a life-threatening condition. Mortality due to liver disease is the only leading cause of death that has steadily increased year on year since the 1970s in the United Kingdom and it remains a major health burden worldwide (Fallowfield 2015). The only therapeutic approaches entail removal of the injurious stimuli (e.g. administration of an efficacious anti-viral therapy) and liver transplantation. Delivering an effective anti-fibrotic therapy is therefore a major unmet clinical need for both chronic and acute liver damage.

Macrophages (Mϕ) play a pivotal role in the inflammatory response in the injured liver. In the liver there are two main populations of Mϕ: (i) resident macrophages (Kupffer Cells, KCs), and (ii) infiltrating macrophages. KCs carry out patrolling functions in the liver sinusoids to phagocytose microbial debris that reach the liver via the sinusoidal capillaries in homeostatic conditions. During the early phases of liver damage, KCs express chemokines such as CCL2 and CCL5, thereby contributing to the recruitment of monocytes from the circulation. The number of KCs decreases during fibrosis; they then repopulate the liver in the recovery phase of self-limiting fibrosis. Infiltrating monocyte derived Mϕ (MDMs) play a major role in the response to liver damage. Infiltrating Mϕ are recruited via the CCR2/CCL2 axis; once in the liver parenchyma, they locate along the fibrotic septa in the early stages of liver fibrosis and may promote fibrosis by releasing factors such as TGF-β, IL1, PDGF and CCL2 that activate hepatic stellate cells and worsen inflammation. This may suggest a detrimental role for Mϕ in progressive fibrosis. However, if Mϕ are depleted at the onset of fibrosis remodelling, the remodelling process fails, and the liver fibrosis persists. It is now widely accepted that macrophages play a dual role in the establishment and resolution of fibrosis.

Due to their role in healing of fibrosis, it has been considered that a macrophage cell therapy could be beneficial for the reduction of CCl₄-induced chronic liver fibrosis. It has been shown that mouse bone marrow-derived macrophages (BMDMs) improve liver fibrosis when injected into a mouse model of chronic liver fibrosis. Similar results have been replicated using human monocyte-derived macrophages (hMDMs) in immunodeficient mouse models of chronic liver fibrosis. Furthermore, a GMP-graded cell culture protocol is currently used to generate hMDMs for autologous transplantation into cirrhotic patients in an ongoing phase II trial (MATCH, Macrophage therapy for liver disease, ISRCTN10368050).

Macrophages are thus a critical part of the human immune system. These monocyte-derived cells play a key role in the control of host homeostasis. Macrophages possess high phagocytic and secretory activities, which bring about their ability to clear dead and altered cells, prompt inflammatory reactions, eradicate pathogens, in addition to the control of overstated immune responses, and mediate tissue repair. Macrophages are antigen-presenting cells, offering T cells antigens derived from engulfed pathogens using HLA II on their surface.

Macrophages may acquire various states, referred to as “polarisation”, which are usually, but simplistically, divided into two main extremes, “pro-inflammatory” (or classically-activated, “M1-like”) and “pro-regenerative” (or alternatively-activated, “M2 like”, anti-inflammatory or anti-fibrotic). However, macrophages may adopt a state between these extremes which may be “unpolarised” or naïve (M0) or point more towards an anti or pro-inflammatory state.

Macrophages acquire a “pro-regenerative” state under the action of various factors in combination, including macrophage colony-stimulating factor (M-CSF), IL-4, IL-13, IL-10 and TGF-β. These macrophages mediate wound healing and tissue regeneration primarily.

It has been documented that impairment of macrophage function or an imbalance between their pro- and anti-inflammatory activities can underpin several diseases and conditions, including autoimmune disorders, cancer, cardiovascular conditions, infections, metabolic conditions and neurodegenerative disorders.

Macrophages as a cell based therapy are an attractive option, since they could be provided in a particular state which is appropriate for the disease or condition to be treated, such as a pro-inflammatory macrophage for use in treating cancer or a pro-regenerative macrophage for treating liver cirrhosis.

Macrophages derived from monocytes (MDMs) are the main source of human macrophages for use in clinical studies, which involves the harvesting of monocytes from a donor, their conversion or macrophage and their reinfusion to the patient. More recently, researchers looking into the potential of allogenic treatment have explored the possibility of using macrophages generated from stem cells, such as induced pluripotent stem cells. However, these cells inherently will retain the characteristics of the donor cell from which the stem cells are derived, which may ultimately limit their universal application or their repeated use. Certain cell markers, such as the Human Leukocyte Antigens (HLAs) will identify these allogeneic cells as “foreign” and mark them for clearance or destruction.

Nevertheless, as macrophages form a critical part of the immune system, it has been thought that attempting to make these cells hypoimmunogenic by downregulating expression of cell markers involved in foreign cell recognition may impair their function. For example, HLA class II molecules are constitutively expressed on professional antigen-presenting cells, such as monocytes and macrophages and are an essential component of their function as antigen-presenting cells. Removing such components to make the resulting macrophage hypoimmunogenic may alter the ways the cells inherently work and indeed polarise. Further, it has not previously been investigated whether macrophages lacking key cell markers can still perform as macrophages and “polarise” to either pro-inflammatory or pro-regenerative macrophages.

Finally, HLAs have been shown to be linked to differentiation of antigen presenting cells. Indeed, it has been recognised that upregulation of MHC molecules on the cell surface, such as those encoded by HLA-DR, occurs upon migration of classical monocytes into tissues, and that these HLA-DR^{hi} monocytes represent an intermediate stage through which cells pass in order to mature into tissue macrophages (Jardine et al., 2021, Immunity, 54:194-196). It is further known that antigen presenting cells (APCs) express MHC molecules in an immature state, but that these are transported to the lysosome and degraded. This transport to the lysosome is reduced upon activation, and transport to the plasma membrane of MHC class II molecules is enhanced, resulting in increased cell surface expression (Chow et al., 2002, Nature, 418:988-993). Therefore, MHC expression appears to be a key requirement for maturation of macrophages, and it is not known how undifferentiated cells lacking MHC expression could produce functional macrophages.

US 2021/0052643 describes macrophages lacking functional expression of MHC genes, and speculatively suggests generating primary monocytes with HLA knockouts, which may be differentiated into macrophages and polarised into an “M1-like” phenotype. No such cells were actually generated, and no other cell sources are contemplated. WO 2023/003775 and Pouyanfard, et al. (2021, Stem Cells., 39(12): 1701-1717) describe iMAC polarisation to M1 or M2 like phenotypes, and suggest that M2 polarised iMACs may be used to treat liver fibrosis, but neither document addresses the generation of hypoimmunogenic cells or the potential effects of knock outs on macrophage development or polarisation.

Lyadova et al. (2022, Cell Biosci. 12(1):96) speculatively suggests that universal iPSCs could be generated by ablating HLA expression, and that said universal iPSCs could be used to generate universal iMACs. Quinsong Ye et al. (2022, Cell Prolif. 2020; 53:e12946) also suggests that hypoimmunogenic iPSCs could be universal. Neither of these documents provides data demonstrating successful production of any functional hypoimmunogenic iPSC derived macrophages, nor any credible suggestion that such cells may actually be suitable for use in therapy.

WO 2016/183041 describes stem cells with modulated expression of MHC I and II, and macrophages derived from cells devoid of MHC II expression. However, no macrophages essentially devoid of both MHC I and II expression are described, nor are the functionality of hypoimmunogenic iMACs or their use in treatment.

### SUMMARY OF THE INVENTION

The inventors are the first to show that hypoimmunogenic stem cells, most notably those with reduced or removed expression of HLA I and/or HLA II can indeed be differentiated into functional macrophages that can then be polarised, and that can be used in the conditions described herein.

This is entirely surprising given the nature of a macrophage and the requirement of wild-type macrophages for HLA surface expression in their function and differentiation. The inventors have demonstrated, for the first time, that polarisation to both inflammatory and anti-inflammatory phenotypes is possible. Thus, it has also been shown that possession of functional HLA molecules is not critical for polarisation.

The present invention relates to a cell population comprising novel polarised and non-polarised macrophages with deficient expression of HLA I and HLA II as described herein. These novel polarised and non-polarised cells may be used in therapy.

The present invention describes stem cells, preferably induced pluripotent stem cells (iPSC) which have differentiated into macrophages which are then polarizable, providing an “off-the-shelf” universal macrophage suitable for use as an allogenic macrophage cell therapy.

The macrophages are preferably hypoimmunogenic due to the knock out or knock down of at least one gene associated with either HLA Class I or HLA class II cell surface molecules. This knock-out or knock-down is evident in the macrophages, but, as demonstrated by the present invention, may be introduced into any progenitor cell as the macrophage is differentiated from the stem cell while maintaining a functionally active macrophage.

Thus, the stem cells (SC) from which the macrophages are differentiated may be genetically modified to knock out or knock down of at least one gene associated with either HLA Class I or HLA class II cell surface molecules. Alternatively, the genetic modification may be carried out on any cell type during the differentiation of the stem cell towards a macrophage, meaning that the cell that is genetically modified may be any cell type ranging for an entirely pluripotent stem cell, through a multipotent stem cell, to a lineage restricted cell, to a progenitor cell.

The stem cell may be any suitable type of stem cell, for example a pluripotent or multipotent stem cell. Pluripotent stem cells (PSC) are cells that have no fixed developmental potential, and are able to differentiate into different cell types. Embryonic stem cells and induced pluripotent stem cells (iPSC) are pluripotent stem cells. Multipotent stem cells can develop into more than one cell type, but are more limited than pluripotent cells; cord blood stem cells and haematopoietic stem cells are considered multipotent. The only selection criteria is that the stem cell chosen must be capable of differentiation into the progenitors of macrophages, including monocytes.

It is preferred that the macrophage generated retain the knock out or knock down of at least one gene associated with either HLA Class I or HLA class II cell surface molecules.

Optionally, the cell population as described here, the SC is genetically modified to knock down or knock out expression of both HLA I and HLA II. In preferable embodiments, the SC is genetically modified to knock out surface expression of all endogenous HLA I and HLA II. According to some embodiments, the SC is genetically modified to knock out expression of the B2M gene and CIITA gene, thus knocking out surface expression of all endogenous HLA I and HLA II.

Optionally, the genetic modification may be a single knock or a double knock out (i.e. both alleles).

In the macrophage of the cell population, expression of the HLA I and/or HLA II is reduced, optionally when compared to a wild type cell, preferably a wild type macrophage. According to some embodiments, surface expression of HLA I and HLA II in the hypoimmunogenic functional macrophages described herein is reduced or abrogated as compared to macrophages differentiated from SCs having wild-type expression of HLA I and HLA II.

The genetic modification may be any suitable technique, of which they are many known to the skilled person. The genetic modification may act on the gene itself or disrupt expression of the gene, by modifying promoters, splice junctions or non-coding RNA. The modification may be conditional, such that it is only applied when a molecule (such as a drug) is applied to the cell. Various nucleases are known that can be exploited to modify the genome, from base editing techniques, prime editing techniques to gene editing. Many nucleases are known—such as Zinc fingers, TALENs and guided nucleases. These may be guided by RNA (“RNA” guided nucleases)—such as the enzymes involved in CRISPR, including but not limited to Cas9, Cas12a, Cas13, Mad7 and the like. Alternatively or additionally, antisense oligonucleotides or siRNA may be employed to knock out or down gene expression.

The genetic modification involves the use of any one or more of the following technologies:
(i) antisense oligonucleotides;
(ii) siRNA or miRNA;
(iii) nuclease editing.

Targeted genome modifications can be achieved using rare cleaving nucleases, such as zinc-finger nucleases (ZFNs), transcription activator-like effector nucleases (TALENs), meganucleases (mns, also termed homing endonucleases) and clustered, regularly interspaced, short palindromic repeat (CRISPR) RNA-guided nucleases.

CRISPR/CAs9 variant technology may include base editing, a genome editing method that efficiently converts one base pair to a different base pair without inducing double-stranded DNA breaks or extensive insertions and deletions. This can introduce precise edits to introduce a stop codon with a high level of efficiency to prevent gene transcription and thereby expression.

Optionally the knock down or knock out to the HLA class I gene is to the Beta-2 microglobulin gene (B2M). Preferably, all endogenous expression of beta-2 microglobulin is knocked out. Preferably, the knockout of B2M knocks out all endogenous expression of HLA class II genes, specifically HLA-A, HLA-B and HLA-C.

Optionally the knock down or knock out to the HLA class II gene is to the CIITA gene. Preferably, all endogenous expression of CIITA is knocked out. Preferably, the knockout of CIITA knocks out all endogenous expression of HLA class II genes, specifically HLA-DP, HLA-DQ and HLA-DR.

It is preferred that the macrophages of the cell population described here are polarised, such that they are not naïve or unpolarised. It has been shown that the macrophages may polarise in either direction, dependent on the choice of polarisation factors applied. Preferred are the application of polarising factors that promote the generation of a “pro-regenerative” macrophage. Such macrophages are suitable for use in therapy with patients that have chronic inflammation with a fibrotic element.

The choice of these cell populations permits the development of an anti-fibrotic therapy with a reduced risk of “rejection” of these cells by the recipient. Preferably the macrophages of the cell population avoid clearance by the immune system.

As described here, according to some embodiments, at least 70%, 75%, 80%, 85%, 90% or 95% of said macrophages in said cell population express at least two of the following markers: CD14, CD45, 25F9 and/or CD206. Such a range includes any values between these exemplified percentages.

As described here, according to some embodiments, at least 80%, 85%, 90% or 95% of said macrophages in said cell population secrete over 10 pg/ml TNF-A, IL-6, IFN-Y, IL-1B and IL-12p70. Such a range includes any values between these exemplified percentages. Optionally these will be expressed up to a desired threshold.

In particular, the present invention provides a cell population comprising a pro-regenerative macrophage derived from a stem cell (SC), wherein said SC is genetically modified to knock down or knock out of expression of one or both of human leukocyte antigen class I (HLA I) and HLA class II (HLA-II), for use in treating a chronic inflammatory condition with a fibrotic element.

Chronic inflammatory organ damage is often associated with fibrosis, such as for example in chronic liver disease. Therefore, a cell population of pro-regenerative therapeutic macrophages with anti-inflammatory and anti-fibrotic functions would be beneficial wherein this could avoid host immune cell clearance itself.

The condition to be treated may be chronic organ damage associated with chronic inflammation.

The condition to be treated may relate to the kidney, liver, or lung. For example, the condition maybe liver damage, kidney damage or lung damage. Optionally the condition is liver cirrhosis.

There is provided a composition comprising a cell population of pro-regenerative macrophages as described herein for therapeutic anti-inflammatory or anti-fibrotic effect.

The uses as described herein may comprise administering an effective amount of the cell population of pro-regenerative macrophages to the subject or patient in need thereof. This administration may be systemic administration, suitably by systemic injection.

The methods employed herein polarizes the stem cell derived macrophages or indeed induced pluripotent stem cell derived macrophage progenitors to a pro-regenerative phenotype and/or away from a pro-inflammatory phenotype.

Hypoimmunogenic stem cells or hypoimmunogenic cells derived from stem cells may be described as cells that have been genetically modified to at least knock down or knock out expression of one or both human leukocyte antigen class I (HLA I) and HLA class II. This may involve knocking down or knocking out any gene or loci related to HLA class I and/or HLA class II. Additionally, it may involve the genetic modulation of other genes.

Human leukocyte antigen (HLA) is a gene system found in humans, which encodes cell-surface proteins involved in the regulation of the immune system. Vertebrates express a similar gene system referred to as major histocompatibility complex (MHCs). The present invention uses genetically modified pluripotent stem cells wherein the expression of various entities HLA-I (HLA-A, HLA-B and HLA-C genes or B2M gene) and/or HLA-II (HLA-DP, DM, DO, DQ, and DR or CIITA) have been either knocked-down or knocked-out. One or more gene or loci is knocked down or out to alter the expression of HLA class I or HLA class II on the cell surface. In preferred embodiments, all endogenous surface expression of HLA class I and II gene products have been knocked out. In particular embodiments, all endogenous surface expression of HLA-A, HLA-B and HLA-C and HLA-DP, HLA-DQ and HLA-DR has been knocked out.

Pro-regenerative macrophages may be interchangeably referred to throughout as anti-inflammatory and/or anti-fibrotic macrophages. Alternatively they may be classed as pro-restorative, “M2-like” or “alternatively activated” macrophages. Thus, the pluripotent stem cell generated macrophages of the present invention have been successfully polarised following their differentiation.

The cell populations of the present invention have a particular use in cell therapy. As they are hypoimmunogenic, they are useful as a universally applicable cell type for transfusion.

Once the stem cells, or progeny/descendent cells have been modified to make them hypoimmunogenic, they may be cultured or treated appropriately to differentiate the cells into macrophages as shown in the Examples. Once the macrophages have been generated, they may be polarised as shown in the Examples. These methods are discussed further herein. In the Examples, the inventors demonstrate that the macrophages can surprisingly still polarise to a pro-regenerative type, despite the absence of key cell surface glycoproteins.

Advantageously, the inventors postulate that transplantation or transfusion of the resulting cell population of generated macrophages into a human patient overcomes immune rejection due to reduced expression of HLA-I and/or HLA-II, whilst still enabling an anti-fibrotic/anti-inflammatory mode of action.

The present invention describes a method of making a cell population of pro-regenerative macrophages derived from hypoimmunogenic stem cells. Suitable hypoimmunogenic stem cells are generated by genetically modifying stem cells, for example.

Genome-editing methods used to target gene sequences essential for HLA-I and/or HLA-II are described in the art which includes the application of CRISPR/Cas9, antisense oligonucleotides, siRNA, zinc fingers, and TALENs

The invention provides a hypoimmunogenic macrophage polarised to a pro-regenerative phenotype. Such may be suitable for use in treating inflammation and/or fibrosis.

The invention also provides a hypoimmunogenic macrophage polarised to a pro-inflammatory phenotype. Such may be suitable for use in oncology treatments.

The hypoimmunogenic macrophages are derived from a hypoimmunogenic precursor or progenitor that is differentiated from a stem cell.

The invention also provides a method for producing hypoimmunogenic functional macrophages, the method comprising:
(a) providing stem cells that are essentially devoid of functional HLA I and HLA II complexes on their surface; and
(b) differentiating the stem cells into hypoimmunogenic functional macrophages.

The invention also provides a cell composition comprising macrophages obtained by the method of the invention.

Surprisingly, the examples demonstrate that functional macrophages can be produced from stem cells essentially devoid of HLA I and HLA II complexes on their surface. Previously it was understood that making macrophages which are hypoimmunogenic may impact their function. Indeed, upregulation and expression of HLA I and II complexes was understood to be a key requirement for macrophage maturation. Accordingly, it was not expected that macrophages could be differentiated from stem cells devoid of HLA expression, and certainly not expected that any such macrophages would retain their normal function.

In a particularly preferred embodiment, the stem cells are pluripotent stem cells, most preferably induced pluripotent stem cells, which are essentially devoid of functional HLA I and HLA II complexes on their surface due to a knockout of both B2M and CIITA. Accordingly, the method provided by the invention may comprise steps of:
a) providing induced pluripotent stem cells that are essentially devoid of functional HLA I and HLA II complexes on their surface wherein expression of both B2M and CIITA has been knocked out; and
(b) differentiating the induced pluripotent stem cells into hypoimmunogenic functional macrophages.

The invention also provides a method for producing polarised hypoimmunogenic functional macrophages, the method comprising:
(a) providing stem cells that are that are essentially devoid of functional HLA I and HLA II complexes on their surface;
(b) differentiating the stem cells into functional macrophages; and
(c) polarising the functional macrophages by contacting them with IL-10.

The invention also provides a method of polarising macrophages derived from a stem cells essentially devoid of functional HLA I and HLA II complexes on their surface to a pro-regenerative phenotype, the method comprising contacting the macrophages with IL-10.

It was not expected that hypoimmunogenic macrophages could be polarised with IL-10. The data described in Example 9 of the present application demonstrate that the functional hypoimmunogenic macrophages have reduced expression of IL-10RA (alpha subunit of the IL-10 receptor). Accordingly, it would be expected that such cells would have reduced capacity to be polarised by exposure to IL-10. In contrast, the data as described in the present application, and particularly in Examples 10 and 11, demonstrate that the hypoimmunogenic functional macrophages may be readily polarised into an M2-like phenotype when exposed to IL-10. Indeed, such cells are polarised with at least the same efficiency as macrophages expressing functional HLA complexes.

All cells referred to herein are preferably human.

In certain embodiments, the invention provides:
1. A cell population comprising a pro-regenerative macrophage derived from a stem cell (SC), wherein said macrophage is genetically modified to knock down or knock out of expression of one or both of human leukocyte antigen class I (HLA I) and HLA class II (HLA-II), for use in treating a chronic inflammatory condition with a fibrotic element.
2. A cell population as provided in embodiment 1 wherein the SC is an induced pluripotent stem cell (iPSC).
3. A cell population as provided in embodiment 1 or 2 wherein said SC or cell derived therefrom is genetically modified to knock down or knock-out one or more genes or loci associated with HLA I and/or HLA II.
4. A cell population as provided in any preceding embodiment wherein said macrophages in said cell population are hypoimmunogenic.
5. A cell population as provided in any preceding embodiment wherein said macrophages in said cell population evade clearance by the immune system.
6. A cell population as provided in any preceding embodiment wherein at least 70%, 75%, 80%, 85%, 90% or 95% of said macrophages in said cell population express at least two of the following markers: CD14, CD45, 25F9 and/or CD206.
7. A cell population as provided in any preceding embodiment wherein at least 80%, 85%, 90% or 95% of said macrophages in said cell population secrete over 10 pg/ml TNF-A, IL-6, IFN-Y, IL-1B and IL-12p70.
8. A cell population as provided in any preceding embodiment wherein said SC or cell derived therefrom is genetically modified to knock down expression of both HLA I and HLA II.
9. A cell population as provided in any preceding embodiment wherein said SC or cell derived therefrom is genetically modified to knock out expression of both HLA I and HLA II.
10. A cell population as provided in any preceding embodiment wherein said gene is a single knock or a double knock out.
11. A cell population as provided in any preceding embodiment wherein the expression of the HLA I and/or HLA II is reduced, optionally when compared to a wild type cell.
12. A cell population as provided in any preceding embodiment wherein the genetic modification involves the use of any one or more of the following technologies:
   (i) antisense oligonucleotides
   (ii) siRNA or miRNA; and/or
   (iii) nuclease editing.
13. A cell population as provided in any preceding embodiment wherein the knock down or knock out to the HLA class I gene is to the Beta-2 microglobulin gene.
14. A cell population as provided in any preceding embodiment wherein the knock down or knock out to the HLA class II gene is to the CIITA gene.
15. A cell population as provided in any preceding embodiment wherein said condition is chronic organ damage associated with chronic inflammation.
16. A cell population as provided in any preceding embodiment, wherein the condition relates to the kidney, liver, or lung.
17. A cell population as provided in any preceding embodiment, wherein the macrophage is anti-inflammatory and anti-fibrotic.
18. A cell population as provided in any preceding embodiment for treating liver fibrosis, optionally liver cirrhosis.

The invention also provides the following embodiments, which may be combined with any other embodiments:

1. A method for producing hypoimmunogenic functional macrophages, the method comprising:
(a) providing stem cells that are essentially devoid of functional HLA I and HLA II complexes on their surface; and
(b) differentiating the stem cells into hypoimmunogenic functional macrophages.

2. The method of embodiment 1, wherein the stem cells are pluripotent stem cells.

3. The method of embodiment 1 or 2, wherein the hypoimmunogenic functional macrophages display at least one or all of the following characteristics: capacity for phagocytosis, ability to polarise to an M1-like phenotype, and ability to polarise to an M2-like phenotype.

4. The method of embodiment 3, wherein capacity for phagocytosis is assessed by determining the percentage of cells phagocytosing in a representative sample of the hypoimmunogenic functional macrophages, and wherein optionally at least 60%, at least 70%, at least 80%, at least 90% or at least 95% of the cells are phagocytosing.

5. The method of any of embodiments 2-4, wherein the pluripotent stem cells are induced pluripotent stem cells (iPSCs).

6. The method of any of embodiments 2-5, wherein the pluripotent stem cells are isolated cells, a cell aggregate or an embryonic body.

7. The method of any of the preceding embodiments, wherein the stem cells are devoid of HLA I and II through knock-out or knock-down of at least one sequence encoding at least one unit in the HLA I and/or the HLA II complex or a transcriptional activator thereof, optionally knock-out or knock-down of the B2M gene, the CIITA gene or a combination thereof.

8. The method of any of the preceding embodiments, wherein the stem cells comprise a genetic modification that prevents surface expression of HLA I and HLA II complexes.

9. The method of embodiment 8, wherein the genetic modification is transformation with a construct encoding an antisense oligonucleotide, siRNA, RNAi or miRNA targeting at least one unit in the HLA I and/or the HLA II complex, or a transcriptional activator thereof, or wherein the genetic modification is mutation or deletion of sequence encoding at least one unit in the HLA I and/or the HLA II complex, or a transcriptional activator thereof.

10. The method of embodiment 8 or 9, wherein the genetic modification results in the expression of a silencing molecule from the genome of the stem cells targeting at least one unit in the HLA I and/or the HLA II complex, or a transcriptional activator thereof.

11. The method of any of embodiments 8-10, wherein the genetic modification comprises gene editing which results in knockout of at least one component of the HLA I and/or HLA II complexes or a transcriptional activator thereof, optionally wherein the gene editing results in knockout of B2M, CIITA or both.

12. The method of any one of embodiments 2-11, wherein the step of differentiating the pluripotent stem cells into hypoimmunogenic functional macrophages comprises a step of differentiating the pluripotent stem cells into embryonic bodies.

13. The method of embodiment 12, wherein the step of differentiating the pluripotent stem cells into embryonic bodies comprises incubating the pluripotent stem cells with at least one or all of BMP4, SCF and VEGF.

14. The method of embodiment 13, wherein the step of differentiating the pluripotent stem cells into embryonic bodies further comprises incubating the cells with Rock Inhibitor (Y-27632).

15. The method of any one of embodiments 2-14, wherein the method comprises a step of differentiating the embryonic bodies into macrophage progenitors.

16. The method of embodiment 15, wherein the step of differentiating the embryonic bodies into macrophage progenitors comprises culturing the embryonic bodies in the presence of M-CSF and/or IL-3.

17. The method of embodiment 16, wherein at least 80% of the macrophage progenitors produced by the step of differentiating the embryonic bodies into macrophage progenitors express at least one or all of CD45, CD14, CD206 and 25F9.

18. The method of any one of the preceding embodiments, wherein the method comprises a step of maturing the macrophage progenitors into functional macrophages.

19. The method of embodiment 18, wherein the step of maturing the macrophage progenitors into functional macrophages comprises culturing the macrophage progenitors in the presence of M-CSF.

20. The method of any one of the preceding embodiments, wherein the method further comprises polarising the macrophages by contacting the macrophages with IL-10.

21. A method of polarising macrophages derived from stem cells essentially devoid of functional HLA I and HLA II complexes on their surface to a pro-regenerative phenotype, the method comprising contacting the macrophages with IL-10.

22. The method of embodiment 20 or 21, wherein the contacting with IL-10 comprises providing IL-10 exogenously as a protein.

23. The method of embodiment 20 or 21 wherein the contacting with IL-10 comprises engineering the functional macrophage to express IL-10.

24. The method of embodiment 23, wherein the functional macrophage is engineered to express IL-10 transiently.

25. The method of embodiment 24, wherein the functional macrophage is engineered to express IL-10 transiently by transfecting the cell with DNA or RNA.

25. The method of embodiment 20 or 21, wherein contacting the macrophages with IL-10 comprises engineering the stem cell to express IL-10 stably.

26. The method of embodiment 25, wherein the stem cell is engineered to express IL-10 stably by introducing a sequence encoding IL-10 into the stem cell, optionally wherein the sequence encoding IL-10 is introduced into the nuclear genome of the stem cell.

27. The method of any preceding embodiment, comprising formulating the hypoimmunogenic functional macrophages in a therapeutic composition.

28. A cell composition comprising macrophages obtained by the method of any of the preceding embodiments.

29. The cell composition of embodiment 28, for use in treating liver injury, optionally wherein the liver injury comprises liver fibrosis, inflammatory liver injury with a fibrotic element, chronic liver injury and/or liver cirrhosis.

30. The cell composition of embodiment 29, wherein the liver injury is liver cirrhosis.

31. A cell population comprising a pro-regenerative macrophage derived from a stem cell (SC), wherein said macrophage is genetically modified to knock down or knock out of expression of one or both of human leukocyte antigen class I (HLA I) and HLA class II (HLA-II), for use in treating a chronic inflammatory condition with a fibrotic element.

32. A cell population of embodiment 31 wherein the SC is an induced pluripotent stem cell (iPSC).

33. A cell population of embodiment 31 or embodiment 32 wherein said SC or cell derived therefrom is genetically modified to knock down or knock-out one or more genes or loci associated with HLA I and/or HLA II.

34. The cell population of embodiments 31-33, wherein the macrophage is genetically modified to knock down or knock out of expression of both HLA I and HLA II.

35. The cell population of embodiment 34, wherein the macrophage is genetically modified to knock out expression of both HLA I and HLA II.

36. The cell population of embodiment 35, wherein the macrophage is genetically modified to knock out endogenous expression of both HLA I and HLA II.

37. The cell population of embodiment 35 or 36, wherein genetically modifying the macrophage comprises knocking out both the beta-2 microglobulin (B2M) and the Class II Transactivator (CIITA) loci.

38. The cell population of any of embodiments 31-37, wherein the chronic inflammatory condition with a fibrotic element is liver fibrosis, optionally liver cirrhosis.

39. The cell population of any of embodiments 31-38, wherein the pro-regenerative macrophage has been polarised to the pro-regenerative phenotype by contacting the macrophage with IL-10.

### DESCRIPTION OF THE FIGURES

FIG. 1. Analysis of pluripotency markers by flow cytometry. Both P3 (A) and P3KO (B) express >90% of TRA-1-60, SSEA4, OCT3/4 and Nanog with <10% expression of SSEA1. The P3 cells are iPSCs which have been generated essentially as described in (Baghbaderani, Syama et al. 2016) which are reprogrammed using umbilical Cord Blood (hUCB) CD34+ cells and episomal plasmids encoding Oct4, Sox2, Klf4, c-Myc and Lin28. The P3KO cells are similar to the P3 cells but contain a knockout in the B2M and CIITA genes which results in no expression of HLA I and HLA II on the surface of the iPSC cells and cells differentiated from them.

FIG. 2. Macrophage differentiation overview. Morphological representation of phase contrast images of the different stages of the differentiation. Pluripotent Stem Cell (PSC) culture displayed a compact cell colony of PSC, this is characterised by cells forming high density clusters Embryonic bodies were generated using suspension culture (Aggrewell 800 or 96 well plate ULA), embryonic bodies differentiation is characterised by an heterogenic morphology within the EB, cell differentiation occurs displaying heterogeneity cell density within the EB. Macrophages are characterised by displaying a granular morphology with low nuclei-to-cytoplasm ratio.

FIG. 3. Flow Cytometry surface marker characterisation of iMACs. iMACs express high levels of CD45, CD14, CD206 and 25F9. A. Average expression of single markers of lot 5 matured iMACs, data plotted as a bar plot. iMACs were derived from both P3 and P3KO pluripotent stem cells. B. Representative flow cytometry dot plot of lot 5 mature iMACs, co-expression of markers was visualised to ensure homogeneous co-expression of markers is achieved.

FIG. 4. Analysis of HLA I and II expression by flow cytometry. P3KO HLA I (Left panel) and HLA II (Right panel) expression is reduced at iPSC, iMACs progenitors and matured iMACs.

FIG. 5. Mature HLA I and II knock out iMACS phagocytose e. coli beads. A. Representative image of iMAC phagocytosis, 120 mins after co-incubation of iMACs (GFP) and E.Coli beads (labelled with a different colour) A shows iMACs derived from P3KO stem cells. Co-localisation of the labelled iMACs and E. coli beads demonstrates phagocytosis. B. Enumerated flow cytometry analysis of % CD14+ve E. coli bead+ve iMACS. C. Representative flow cytometry density-plots, showing CD14+ve, E. coli bead+ve iMACs.

FIG. 6. Mature HLA I and II knock out iMACs can be polarised towards M1 and M2 phenotypes. A. Cytokine secretion analysis on iMACs stimulated with IFN-y and LPS, shows >1000-fold upregulation in the secretion of inflammatory cytokines TNF-A and IL-6, compared to M0 macrophages. B. Enumerated flow cytometry analysis shows that IFN-Y and LPS-treated iMACs upregulate the M1 marker, CD86 and down-regulate the M2 markers CD206, 25F9 and CD163. C. IL-10 stimulated iMACs have increased expression of the M2 markers CD206, 25F9 and CD163. Data is plotted in bar charts and represents an average of 2 differentiations at lot 5±SD.

FIG. 7. Cytokine secretion profile of steady state iMACS. Conditioned medium was collected over a 24 hour period and assessed for the presence of inflammatory cytokines. Steady state iMACs do not secrete the inflammatory cytokines TNF-A, IL-6, IFN-Y, IL-1B and IL-12p70.

FIG. 8. IL-10RA transcriptional expression levels in non-polarised and IL-10 polarised HLA-deficient iMACs and in hMDMs. iMACs or hMDMs were unpolarised or polarised using IL-10 as described in Example 3. The expression of IL-10RA was measured at the transcriptional level using RNA sequencing. iMACs had reduced expression of IL-10RA compared to hMDMs of the same polarisation state.

FIG. 9. Surface expression levels of CD206 & CD80 in of HLA-deficient iMACs and in hMDMs in various polarisation conditions, as measured by flow cytometry. A tabulates the percentage of cells positive for CD206 and CD80 in unpolarised, M1 polarised cells (IFN-γ and LPS), and cells polarised to an M2 phenotype using IL-10 or IL-4 and IL-13. B and C plot the mean fluorescent intensity (MFI) for CD206 (B) and CD80 (C) of cells in each polarisation state. The MFI shown corresponds to MFI of live single cells in the full stain samples (stained with all antibodies) minus the MFI of live single cells from the corresponding fluorescence minus one (FMO). Each data point corresponds to mean of two technical duplicates of three independent iMAC differentiations and three different hMDM donors.

FIG. 10. Cytokine secretion levels of iMACs and of hMDMs in various polarisation conditions. Conditioned medium was collected over a 24 hour period and the concentration of CCL26 (A), CCL2 (B), IL-8 (C) and TNF-α (D) was measured. iMACs and hMDMs were unpolarised, or were polarised with IFN-γ and LPS (M1), IL-10 (M2) or IL-4 and IL-13 (M2).

FIG. 11. Phagocytic capability of HLA-deficient iMACs & of hMDMs in various polarisation conditions. Percentage of phagocytosing live single cells, as measured by the method described in Example 4, using E. coli beads. The MFI shown here corresponds to the MFI of live single cells in the full stain samples minus the MFI of live single cells from the corresponding FMO. Each data point corresponds to the average of two technical duplicates of two independent iMAC differentiations and two different hMDM donors.

The present invention will now be described further with reference to the following headed sections. Any features under any of the sections may be combined with any of the aspects or embodiments of the invention in any workable order.

### DETAILED DESCRIPTION

Provided herein are cell populations comprising polarised macrophages derived from SCs, optionally PSCs, optionally iPSCs. (herein interchangeably called “hypoimmunogenic macrophages”, “induced macrophages” or simply “iMACS”). Ideally, the stem cells (such as induced pluripotent stem cells) or cells derived therefrom have been made hypoimmunogenic by genetic modification such as knocking down or knocking out one or both of any suitable HLA class I and class II genes. The polarised macrophage is preferably pro-regenerative.

### HLA Class I and II

In humans, the Major Histocompatibility Complex (“MHC”) Genes are referred to as the Human Leukocyte Antigen (“HLA”) genes. This includes HLA I and HLA II genes. It is understood that when “HLA” genes are referred to herein, equivalent genes in cells of other animals (e.g., “MHC” genes) may be similarly altered to produce similar outcomes.

The human leukocyte antigen (HLA) system is an integral part of the immune system and is controlled by genes located on chromosome 6. These encode cell surface molecules which present antigenic peptides to the T-cell receptor (TCR) on T cells. HLA class I and class II form complexes with antigen peptides, interact with T cell receptors (TCRs) of CD8+ T cells and CD4+ T cells, and present antigens. This HLA-peptide-TCR interaction initiates the antigen-specific adaptive immune response. They also act as a ligand for T cells and NK cells for the recognition of self/non-self-components. T cells recognize cells that express non-self HLA, whereas NK cells recognize cells that do not express self HLA. Based on these mechanisms, non-self-cells are eliminated from the body.

HLA molecules that present antigen are divided into 2 main classes: Class I HLA molecules (HLA I) and Class II HLA molecules (HLA II).

Class I molecules are generally observed as transmembrane glycoproteins on the surface of any nucleated cell. Such molecules generally consist of an alpha heavy chain bound to a beta-2 microglobulin molecule. This heavy chain consists of 2 peptide-binding domains, an immunoglobulin-like domain, and a transmembrane region with a cytoplasmic tail. The genes at HLA-A, HLA-B, and HLA-C loci encode the heavy chain of the class I molecule. Some class I MHC genes encode non-classical molecules, such as HLA-G, HLA-E, HLA-L, HLA-J, HLA-K and HLA-H. HLA class I proteins associate with P2-microglobulin, which unlike the HLA proteins is encoded by a gene on chromosome 15.

In human nucleated cells, Class I HLAs (A, B, and C) present peptides from inside the cell. For example, if the cell is infected by a virus, the HLA system brings fragments of the viral proteins to the cell surface so that the cell can be targeted by the immune system. In general, these particular peptides are small, such as about 8-10 amino acids in length. “Foreign” antigens presented by HLA class I attract T CD8-positive (or cytotoxic T-cells) that destroy the cells displaying the antigen on their surface. The specificity for peptide binding on the HLA class I molecule peptide binding cleft is determined by the α chain. Recognition by CD8+ T cells of the peptides presented by the HLA class I molecule mediates cellular immunity.

However, from an allogeneic source, the HLA class I glycoprotein itself constitutes a foreign antigen. The recognition of non-self HLA class I protein is a major obstacle in using allogenic PSCs or cells derived therefrom for therapy. There exists a need for a more effective macrophage-based therapies that are not impeded by potential rejection and/or clearance.

Designing cells that are deficient in HLA Class I molecules relies upon knocking down or out at least one of the genes of the HLA class I molecules. For Example, U.S. Pat. No. 6,514,752 describes how the β2-microglobulin gene is inactivated for reducing or eliminating the expression of functional Class I MHC antigens. The resulting cells may be used as universal donor cells.

Class II molecules are usually present only on professional antigen-presenting cells (APC), including macrophages, but expression can be induced in some nucleated cells by interferon (IFN)-gamma. They are present on other professional APC cells such as B cells dendritic cells, Langerhans cells, thymic epithelium, and activated T cells. Class II molecules consist of 2 polypeptide (alpha and beta chains); each chain having a peptide-binding domain, an Immunoglobulin-like domain, and a transmembrane region with a cytoplasmic tail. Genes in the HLA-DP, -DQ, or -DR region of chromosome 6 encode both alpha and beta chains. As a whole, there are five isotypes of the class II HLA protein designated as HLA-DM, -DO, -DP, -DQ, -DR.

Class II HLAs (DP, DM, DO, DQ, and DR) present antigens from outside of the cell to T-lymphocytes. Thus, the antigens presented by class II peptides are derived from extracellular proteins.

In order to knock down to out HLA expression in its entirety, both HLA-I and HLA-II must be knocked out/down. Since the region of the HLA-I/II gene is very large, direct gene editing to achieve this is difficult. Thus, researchers have looked for ways to remove associated genes such that the HLA are inoperative or inactive. These are discussed further below.

An HLA gene or genes can be knocked out in the SCs, the cells derived or differentiated from the stem cells or resultant macrophages. Any combination of knock-downs or knock-outs is envisaged herein.

As a common subunit, the B2M gene, may be targeted to suppress the expression of HLA-class I. Thus, this means to eradicate effective HLA class I on the cell surface, the B2M gene is knocked down or out. Thus, the expression of B2M may be reduced when compared to wild type expression.

CIITA is a human gene which encodes a protein called the class II, major histocompatibility complex, transactivator. CIITA is a transcriptional activator of HLA Class II gene expression. CIITA mRNA can only be detected in HLA class II-positive cells. This suggests that expression of HLA class II genes is to a large extent under the control of CIITA. Alternatively or additionally, the CIITA gene can also be knocked out or knocked out to suppress the expression of HLA class II genes. Thus targeted knock out or knock down of the CIITA gene may be used for the suppression of HLA-II.

The RFXANK gene provides instructions for making a protein that assists with control of transcription of HLA class II genes. Transcription is the first step in the production of proteins, and RFXANK is critical for the production of HLA class II proteins from these genes. Thus, knocking this gene down or out can impact the expression of HLA class 11.

Thus, in order to knock out or knock down HLA class I and/or II, it may be necessary to knock down or out other related entities which are responsible for transcription of these cell surface molecules.

A combination of B2M and CIITA targeting may lead to the formation of cells in which HLA is completely knocked down or out.

In some embodiments, targeting of components of the HLA Class I and II complexes, or transcriptional activators thereof, leads to the formation of cells which are essentially devoid of functional HLA I or II complexes on the cell surface. In some embodiments, targeting of components of the HLA Class I and II complexes, or transcriptional activators thereof, leads to the formation of cells devoid of endogenous surface expression of HLA class I or II. In particular embodiments, the cells are devoid of endogenous surface expression of HLA-A, HLA-B and HLA-C and HLA-DP, HLA-DQ and HLA-DR gene products. Various methods for knocking down or out the relevant genes are described in the literature, for example T. Deuse et al., Nature Biotechnology 37, 252-258 (2019),; Han, X. et al., PNAS May 21, 2019 116 (21) 10441-10446; and Liu et al, Front. Immunol., 2 Jun. 2017, Sec. Alloimmunity and Transplantation.

CRISPR technology can be used to target coding sequences of genes essential for HLA Class I and HLA Class II functionality. Guide RNA (gRNA) can be designed and synthesized for the appropriate gene or loci. The gRNA may then then delivered to PSCs, for example by electroporation. Following delivery, viable single cells which express relevant cell surface markers can be isolated using flow cytometry or magnetic bead-based selection columns to select for edited PSCs. Single cells can then be expanded into colonies and tested for CRISPR editing by isolating the DNA, performing PCR, and performing sequencing using standard techniques.

Accordingly, in any embodiment of the invention, the stem cells comprise a genetic modification that prevents surface expression of HLA I and HLA II complexes. The genetic modification may be transformation with a construct encoding an antisense oligonucleotide, siRNA, RNAi or miRNA targeting at least one unit in the HLA I and/or the HLA II complex or a transcriptional activator thereof. The genetic modification may be mutation or deletion of sequence encoding at least one unit in the HLA I and/or the HLA II complex, or a transcriptional activator thereof. The genetic modification may be gene editing of sequence encoding at least one unit in the HLA I and/or the HLA II complex, or a transcriptional activator thereof, leading to deletion, mutation or disruption of the sequence.

### Knock Down or Knock Out

A gene is “knocked down” when the gene is made partially inoperative such that is expresses a reduced amount of functional protein. Gene knockdown is a technique by which the expression of one or more genes is reduced. This reduction can occur through any method known in the art, such as genetic modification as discussed further below.

A gene is “knocked out” when the gene is made inoperative such that it does not expresses a functional protein. Genes knockout can be achieved by any method known in the art, such as genetic modification. This may includes genetic modification techniques such as causing base editing and frameshift mutations through DNA breaks and repair through mechanisms such as non-homologous end joining (NHEJ) or homologous recombination, or through the use of site-specific nucleases such as zinc fingers and transcription activator-like effector nuclease (TALENs) and CRISPR/Cas9. Those skilled in the art appreciate that residual expression levels may exist after the genes have been knocked out.

As described herein, the knock out or knock down can be of one or both alleles, such that a single or double knock out/down is achieved. These may also be referred to as heterozygous or homozygous gene modifications.

Provided herein are SCs (such as iPSCs), cells derived from stem cells and macrophages in which the knock down or knock out of HLA or HLA-related genes (such as the Beta-2 microglobulin “B2M” gene) has been performed. In some cells, CRISPR-Cas9 genome editing may have been performed to selectively knockout or knockdown any one or more of the HLA I genes. Alternatively, a different method of gene editing has been performed to knock down or knock out the gene. Any suitable method or mechanism of knockdown/knockout may be performed to reach the cells of the present invention. In preferred embodiments, the SCs are essentially devoid of functional HLA I and HLA II expression. In preferred embodiments, the SCs are devoid of endogenous surface expression of HLA class I and II gene products.

In preferred embodiments, the stem cells are iPSCs wherein both B2M and CIITA have been knocked out.

### Hypoimmunogenic Stem Cells or Hypoimmunogenic Stem Cell Derivatives

For cell therapies, the developments of “universal” or hypoimmunogenic SCs with techniques such as base-editing or gene-editing by knocking out immune-related genes is under scrutiny. Such cells have been prepared in several ways, for example B2M-knockout PSCs have been prepared which did not harbour human leukocyte antigen (HLA)-expressing class I cells. Alternatively, major HLA class I molecules have been knocked out, and other genes were knocked in SCs using CRISPR/Cas9 gene editing. It is necessary to develop SCs that do not express HLA class I and/or class II molecules even after differentiation into specific cell lineages. These universal or hypoimmunogenic SCs can be used for clinical therapy in patients with different types of HLA class I and class II molecules.

Several researchers have produced SCs in which B2M is knocked out, the strategy being that B2M protein forms a heterodimer with HLA class I proteins and is required for HLA class I expression on the cell. Thus, knocking out the B2M gene can restrict an immune response from cytotoxic CD8+ T cells by depleting all HLA class I molecules (HLA-A, -B, -C, -E, -F and -G). Approaches to knocking out this gene range from the use of transcription activator-like effector nuclease (TALEN) engineering in iPSCs through RNA interference (RNAi) engineering in iPSCs and CD34+ SC progenitor cells.

Reports are available on the generation of SCs that lack HLA Class I and Class II and their differentiation into epithelial cell types (Petrus-Reurer S, et al, Stem Cell Reports. 2020 Apr. 14; 14(4):648-662). In these pluripotent cells, single-knockout beta-2 microglobulin (SKO-B2M), class 11 major histocompatibility complex transactivator (SKO-CIITA) and double-knockout (DKO) cell lines were generated. These were differentiated into cell lines lacking either surface human leukocyte antigen class I (HLA-1) or HLA-II, or both. The authors found that activation of CD4+ and CD8+ T-cells was markedly lower by DKO cells, while natural killer cell cytotoxic response was not increased. The gene editing in this study was performed by CRISPR/cas 9 gene editing.

Suitable hypoimmunogenic SCs are therefore documented in the literature (such as Ye, Q, Sung, T-C, Yang, J-M, Ling, Q-D, He, Y, Higuchi, A. Generation of universal and hypoimmunogenic human pluripotent stem cells. Cell Prolif. 2020; 53:e12946.) and the skilled person would be aware of suitable methods to either generate these or indeed purchase them from suitable cell suppliers. A cell which is capable of differentiating into a blood cell, particularly myeloid cell line differentiation is desirable.

In one embodiment of the present invention, knocking out the HLA class I gene/loci is achieved by knocking out B2M gene which is a component of the Class I molecules. In another embodiment, knocking out the HLA class I gene/loci is achieved by knocking out genes (HLA-A, HLA-B and HLA-C) individually. In another embodiment, reduced expression of class I glycoproteins is achieved by knocking out processing enzymes which prevent surface expression of the HLA I glycoproteins.

In one embodiment, knocking out the HLA class II gene/loci is achieved by knocking out a master transcription factor such as CIITA gene or the RFANX gene that are required for Class II gene expression.

In some embodiments, the stem cell is a pluripotent stem cell. In preferred embodiments, the pluripotent stem cell is an induced pluripotent stem cell (iPSC). In some embodiments, the stem cell is an isolated cell, a cell aggregate or an embryonic body.

In particular embodiments, knocking out HLA class I gene/loci knocks out all endogenous surface expression of HLA-A, HLA-B and HLA-C gene products. In particular embodiments, knocking out HLA class I gene/loci results in cells essentially devoid of surface expression of HLA-A, HLA-B and HLA-C gene products. In some embodiments, this is achieved by knocking out the B2M gene.

In particular embodiments, knocking out HLA class II gene/loci knocks out all endogenous surface expression of HLA-DR, HLA-DQ and HLA-DP gene products. In particular embodiments, knocking out a transcription activator of the HLA II complex knocks out all endogenous surface expression of HLA-DR, HLA-DQ and HLA-DP gene products. In particular embodiments, knocking out HLA class II gene/loci or a transcription activator of the HLA II complex results in cells essentially devoid of surface expression of HLA-DR, HLA-DQ and HLA-DP gene products. In some embodiments, this is achieved by knocking out the ClITA gene.

In preferred embodiments, both B2M and CIITA are knocked out in the stem cell.

### Differentiation of Stem Cells to Macrophage Progenitors and Macrophages

Methods are available in the art for the differentiation of stem cells to macrophage progenitors, such as monocytes and indeed macrophages themselves. Suitably, this is discussed in van Wilgenburg et al, PLOS ONE, August 2013, Volume 8, Issue 8, e71098. The methods defined therein are entirely feeder-free and serum-free culture conditions to produce very consistent yields across different stem cells types. Particularly, the disclosed methods generated good yields of monocytes, which are a macrophage precursor or progenitor cell. A cell culture medium suitable to differentiate such monocytes into macrophages is discussed below.

In some embodiments, the stem cells are differentiated to macrophage progenitors in a stepwise differentiation protocol. For example, the method may comprise a step of differentiating the pluripotent stem cells into embryonic bodies. In some embodiments, this step may comprise dispensing a single cell suspension of the stem cells into low attachment plates or wells, and incubating with BMP4, SCF and/or VEGF. The cells may be further incubated with Rock Inhibitor (Y-27632). In certain embodiments, the step of differentiating the pluripotent stem cells into embryonic bodies comprises incubating the pluripotent stem cells with at least one or all of BMP4, SCF, VEGF and Rock Inhibitor (Y-27632), such as BMP4 and SCF, BMP4 and VEGF, BMP4 and Y-27632, SCF and VEGF, SCF and Y-27632, VEGF and Y-27632, BMP4 and SCF and VEGF, BMP4 and VEGF and Y-27632, BMP4 and SCF and Y-27632, SCF and VEGF and Y-27632 or BMP4, SCF, VEGF and Y-27632. In some embodiments, the method further comprises a step of differentiating embryonic bodies into macrophage progenitors. In some embodiments, this step comprises culturing the embryonic bodies vertically in low adherence flasks in the presence of M-CSF and/or IL-3. In some embodiments, at least 70% or at least 80% of the macrophage progenitors produced by the step of differentiating embryonic bodies into macrophage progenitors express CD45, CD14, CD206 and 25F9.

In some embodiments, the method comprises a step of maturing the macrophage progenitors into functional macrophages. This step may comprise culturing the macrophage progenitors in the presence of M-CSF. In some embodiments, at least 70% or at least 80% of the functional macrophages produced by the step express CD45, CD14, CD206 and 25F9. In some embodiments, at least 60%, at least 70%, at least 80%, least 90% or at least 95% of the cells are demonstrated to be capable of phagocytosis, or are demonstrated to be phagocytosing. Capacity for phagocytosis may be measured at described herein, such as in Example 4. In preferred embodiments, maturation is deemed complete when at least 95% of the cells in a sample are phagocytosing.

In preferred embodiments, the method comprises steps of:
(a) providing induced pluripotent stem cells essentially devoid of functional HLA I and HLA II expression by knocking out or down the expression of at least one sequence encoding at least one unit in the HLA I and/or the HLA II complex, or a transcriptional activator thereof, in the induced pluripotent stem cells;
(b) differentiating the induced pluripotent stem cells into embryonic bodies, comprising incubating with BMP4, SCF, VEGF and optionally Rock Inhibitor (Y-27632);
(c) differentiating embryonic bodies into macrophage progenitors, comprising culturing the embryonic bodies in the presence of M-CSF and IL-3; and
(d) maturing the macrophage progenitors into functional macrophages, comprising culturing the macrophage progenitors in the presence of M-CSF. In another preferred embodiment, the induced pluripotent stem cells essentially devoid of functional HLA I and HLA II expression are produced by knocking out both B2M and CIITA in the genome of the iPSC cells.

Therefore, in a preferred embodiment, the method comprises steps of:
(a) providing induced pluripotent stem cells essentially devoid of functional HLA I and HLA II expression by knocking out B2M and CIITA in the genome of the induced pluripotent stem cells;
(b) differentiating the induced pluripotent stem cells into embryonic bodies, comprising incubating with BMP4, SCF, VEGF and optionally Rock Inhibitor (Y-27632);
(c) differentiating embryonic bodies into macrophage progenitors, comprising culturing the embryonic bodies in the presence of M-CSF and IL-3; and
(d) maturing the macrophage progenitors into functional macrophages, comprising culturing the macrophage progenitors in the presence of M-CSF.

In preferred embodiments, the induced pluripotent stem cells essentially devoid of functional HLA I and HLA II expression are devoid of endogenous surface expression of HLA I and II.

In preferred embodiments, the method also includes formulating the cells into a pharmaceutical composition. In preferred embodiments, the invention provides cells or a composition generated by the above method, and their use in therapy, particular in the treatment of liver fibrosis, preferably liver cirrhosis.

### Cell Culture Medium

If the cells produced by the differentiation of the stem cells are macrophage precursors such as monocytes, there is already described methodology for culturing monocytes and monocyte precursors into macrophages, such as disclosed in co-pending application WO2021/240162.

Suitably the methods comprise a step of culturing monocytes or precursors in a suitable medium for 3-5 days to produce macrophages. Suitably the monocytes may be cultured in medium within plates or within cell culture cell culture bags. Suitably the medium is suitable for generating macrophages from monocytes. Suitably the medium is a T-cell medium. Suitably the medium may be selected from: X-Vivo 10, X-Vivo 15, TexMACS, AIMv, RPMI, DMEM, and DMEM/F12. Suitably the medium is TexMACS (Miltenyi).

Suitably the medium is serum-free. Suitably the medium is xenoprotein-free. Suitably the medium is GMP-compliant.

Suitably the medium may contain one or more factors. Suitable factors include growth factors, polysaccharides, cytokines and chemokines. Suitable factors may include: MCSF, GM-CSF. Suitably therefore the factors are growth factors. Suitably, the one or more factors are GMP-compliant. In one embodiment, the medium may comprise one or more growth factors may include MCSF or GM-CSF. Monocytes are most commonly cultured with either MCSF or GM-CSF. Culturing monocytes with GM-CSF skews them towards a “pro inflammatory phenotype” whereas culturing monocytes with MCSF skews them towards an “pro-regenerative phenotype”. However, culturing monocytes with both MCSF and GM-CSF is an unorthodox approach. In other embodiments, the one or more growth factors does not include the combination of MCSF and GM-CSF. Thus, if M-CSF is used as the growth factor to generate macrophages in any of the methods of the invention, it may be preferred that GM-CSF is not also used to generate the macrophages from monocytes. This applies to the step of culturing monocytes until macrophages are generated.

Suitably the medium contains MCSF (macrophage colony stimulating factor) otherwise known as CSF-1. Suitably the MCSF may be recombinant MCSF, suitably recombinant human MCSF.

### Polarisation

The generation of macrophages for the cell population used in the invention may comprise a further step of polarisation of the macrophages generated from the pluripotent stem cells. Suitably the step of polarising the macrophages takes place after the macrophages have been generated or differentiated but before isolation or formulation of the macrophages. Suitably any of the methods or uses of the invention may comprise such an additional step.

The invention also provides a method for producing polarised hypoimmunogenic functional macrophages, the method comprising:
(a) providing stem cells that are that are essentially devoid of functional HLA I and HLA II complexes on their surface;
(b) differentiating the stem cells into functional macrophages; and
(c) polarising the functional macrophages by contacting them with IL-10.

The invention also provides a method of polarising macrophages derived from a stem cells essentially devoid of functional HLA I and HLA II complexes on their surface to a pro-regenerative phenotype, the method comprising contacting the macrophages with IL-10.

Suitably the step of polarising macrophages may comprise the addition of one or more polarising factors to the medium. Suitably the one or more polarising factors may produce pro-regenerative macrophages. Suitably the pro-regenerative phenotype are generated by polarisation with various factors as explained below. Pro-regenerative macrophages may be described as “M2-like”. Pro-regenerative macrophages may promote tissue remodelling and regeneration after inflammation is resolved.

Suitably the pro-regenerative phenotype is pro-restorative. Pro-restorative macrophages may promote the resolution of inflammation, for example through the secretion of anti-inflammatory cytokines such as IL-10.

Suitably in order to produce pro-regenerative macrophages the one or more polarising factors include: IL10, IL4, IL13, and poly(I:C). The polarising factor may be IL4+IL13. Alternatively, the polarising factor is IL10. Alternatively, the polarising factor is poly(I:C).

Suitably during the polarisation step, M-CSF is also present in the medium (Monocyte Colony Stimulating Factor, also known as CSF1, Colony Stimulating Factor 1).

Suitably a method comprising a step of polarising the macrophages produces polarised macrophages. Suitably a method comprising a step of polarising the macrophages produces polarised pro-regenerative macrophages.

There is provided a polarised pro-regenerative macrophage produced by such methods.

In one embodiment, there is provided an ex vivo generated polarised macrophage having an anti-inflammatory and anti-fibrogenic phenotype. Anti-fibrogenic macrophages prevent and/or reverse fibrosis. Fibrosis refers to the deposition of excess extracellular matrix and connective tissue following tissue damage, resulting in scarring. Anti-fibrogenic macrophages may resolve fibrosis by the secretion of anti-fibrogenic molecules, such as matrix metalloproteinases (MMPs). Moreover, the secretion of anti-inflammatory cytokines such as IL-10 may resolve inflammation to prevent fibrosis.

In one embodiment, there is provided a population of said polarised macrophages.

In one embodiment, there is provided a cell culture bag comprising said polarised macrophages or a population thereof.

In some embodiments, the polarising factor is provided exogenously as a protein. In other embodiments, the step of polarising macrophages comprises engineering the stem cell or macrophage to express the polarising factor. In particular embodiments, the macrophage may be engineered to express the polarising factor transiently, for example by transient transfection with DNA or RNA. In other embodiments, the stem cell may be engineered to express the polarising factor stably. For example, the stem cell may be engineered by introducing a sequence encoding the polarising factor into the genome of the stem cell.

In preferred embodiments, the polarising factor is IL-10. In preferred embodiments, the polarised macrophages have a pro-regenerative and/or pro-restorative phenotype. In other embodiments, the polarising factor is IFN-γ.

In preferred embodiments, the method for producing polarised hypoimmunogenic macrophages comprises:
(a) providing stem cells essentially devoid of functional HLA I and HLA II expression by knocking out or down the expression of at least one sequence encoding at least one unit in the HLA I and/or the HLA II complex, or a transcriptional activator thereof, in the stem cells;
(b) differentiating stem cells into embryonic bodies, comprising incubating with BMP4, SCF, VEGF and optionally Rock Inhibitor (Y-27632);
(c) differentiating embryonic bodies into macrophage progenitors, comprising culturing the embryonic bodies in the presence of M-CSF and IL-3;
(d) maturing the macrophage progenitors into functional macrophages, comprising culturing the macrophage progenitors in the presence of M-CSF; and
(e) polarising the functional macrophages by contacting them with IL-10.

In preferred embodiments, the method also includes formulating the cells into a pharmaceutical composition. In preferred embodiments, the invention provides cells or a composition generated by the above method, and their use in therapy, particular in the treatment of liver fibrosis.

Macrophages Derived from Genetically Modified SCs or Genetically Modified Progenitor Cells.

Suitably, the invention relates to a cell therapy product, which may be used for treating liver disease. Liver disease include liver cirrhosis, acute liver injury, chronic liver injury, and acute-on-chronic liver failure. Liver disease also includes liver fibrosis, preferably liver cirrhosis.

Suitably, the invention relates to a cell therapy product for inflammatory organ damage based on SC-derived macrophages genetically modified to reduce or remove HLA I and/or HLA II expression that has been polarised to a pro-regenerative form or phenotype.

Suitably, the generated macrophage has a pro-regenerative or pro-restorative form or phenotype; this may also be termed an M2 phenotype and is anti-inflammatory and anti-fibrotic.

### Functional Hypoimmunogenic Macrophages

The invention provides a method for producing hypoimmunogenic functional macrophages. The invention also provides hypoimmunogenic functional macrophages produced by the method of the invention. According to some embodiments, the hypoimmunogenic functional macrophages produced by the method of the invention from induced pluripotent stem cells (iPSCs) may be termed hypoimmunogenic functional “iMACs”.

The functional hypoimmunogenic macrophage is essentially devoid of functional HLA I and HLA II expression on its surface. This may be achieved by knocking out or down the expression of at least one sequence encoding at least one subunit in the HLA I and/or the HLA II complex. Alternatively, this may be achieved by knocking out or down the expression of at least one sequence encoding at least one subunit in the HLA I and/or transactivator of the HLA II complex, such as the B2M gene and/or the CIITA gene, respectively. According to some embodiments, the functional hypoimmunogenic macrophage is essentially devoid of functional HLA I and HLA II expression on its surface, optionally through knock-out of at least one unit of the HLA I complex and at least one transactivator of the HLA II transcription complex, optionally wherein the knock out is of the B2M and CIITA genes. As described herein, the knock out or knock down leading to the lack of functional HLA I and HLA II expression on the functional hypoimmunogenic macrophage is performed in the stem cell (e.g. the iPSC cell) which the macrophage is derived from. Without wishing to be bound by theory or mechanism, differentiating macrophages from stem cells (e.g. iPSCs) which have (i) a knock out or knock down of at least one unit of HLA I and; (ii) a knock out or knock down of at least one unit of HLA II or of a transactivator of HLA II, e.g. knock out of B2M and CIITA, such that the resulting macrophages are essentially devoid of HLA I and HLA II on their surface may result in reduced expression of IL-10RA by the resulting hypoimmunogenic functional macrophages.

In preferred embodiments, the functional hypoimmunogenic macrophage may demonstrate capacity for phagocytosis. This may be determined by measuring the percentage of functional hypoimmunogenic macrophages in a representative sample phagocytosing, for example by using labelled E. coli beads as described in Example 4. In some embodiments, at least 60%, at least 70%, at least 80%, at least 90% or at least 95% of the cells in the sample are phagocytosing. In preferred embodiments, at least 95% of the cells in the sample are phagocytosing. In particular embodiments, M2-polarised functional hypoimmunogenic macrophages that have been polarised by contacting the macrophages with IL-4 and IL-13 may have reduced phagocytic capacity compared to unpolarised functional hypoimmunogenic macrophages or M2-polarised functional hypoimmunogenic macrophages that have been polarised by contacting the macrophages with IL-10.

In some embodiments, the hypoimmunogenic functional macrophages may have reduced expression of IL-10RA compared to human monocyte derived macrophages. In particular, hypoimmunogenic functional macrophages may have reduced transcriptional expression of IL-10RA compared to human monocyte derived macrophages of the same polarisation state. Unpolarised hypoimmunogenic functional macrophages may have reduced expression of IL-10RA compared to unpolarised human monocyte derived macrophages. The hypoimmunogenic functional macrophage may have reduced expression of IL-10RA protein on the cell surface. The hypoimmunogenic functional macrophage may have reduced expression of IL-10RA mRNA.

In some embodiments, the hypoimmunogenic functional macrophage may express comparable levels of macrophage surface markers as human monocyte derived macrophages of the same polarisation state. In particular embodiments, the expression of CD80 or CD206 on the surface of the hypoimmunogenic functional macrophages may be essentially the same as hMDMs of the same polarisation state. In other embodiments, M2 polarised hypoimmunogenic functional macrophages may express greater levels of CD206 on the cell surface than M2 hMDMs. In other embodiments, M1 polarised hypoimmunogenic functional macrophages may express greater levels of CD80 on the cell surface than M1 hMDMs.

In some embodiments, the hypoimmunogenic functional macrophage may secrete comparable levels of cytokines to hMDMs of the same polarisation state. In particular embodiments, the hypoimmunogenic functional macrophage may secrete similar levels of CCL26, CCL2, IL-8 and/or TNF-α compared to hMDMs. In particular embodiments, the hypoimmunogenic functional macrophage may secrete the same level of CCL26 and/or TNF-α compared to hMDMs of the same polarisation state.

In preferred embodiments, the hypoimmunogenic functional macrophage: has at least one unit of the HLA I complex or transactivator of the HLA II complex knocked down or out; has reduced expression of IL-10RA compared to human monocyte derived macrophages of the same polarisation state; secretes similar levels of CCL26, CCL2, IL-8 and TNF-α compared to hMDMs of the same polarisation state; expresses CD45, CD14, CD206 and 25F9; and is capable of phagocytosis.

### Inflammatory Conditions and Treatment

The cell population of hypoimmunogenic macrophages of the present invention are for use in treating a chronic inflammatory condition with a fibrotic element in a subject. As defined above, treating here can mean preventing, reducing or removing inflammation/fibrosis. For example, the cell population maybe administered to a subject at an acute inflammation stage with the aim of preventing a chronic inflammatory condition with a fibrotic element. The cell population may also be administered to a subject at a chronic inflammation stage with the aim of preventing/reducing chronic fibrosis.

Suitably an acute disease or injury may be classed as a disease or injury with an onset of less than 24 weeks from cause. Suitably a chronic disease may be classed as a disease or injury which has persisted for more than 6 months. Suitably an acute-on chronic disease may be classed as a disease or injury with an onset of less than 24 weeks from cause in a patient that already has a chronic disease that has persisted for more than 6 months. Suitably, the cell populations of the present invention maybe administered to a subject with an acute occurrence to prevent transition to or increase of chronic inflammation and fibrosis.

Suitably the subject may be in need of treatment. Suitably therefore the subject may have a disease, or be at risk of developing a disease. Suitably the subject may display one or more symptoms of a disease.

Suitably, the condition is chronic organ damage associated with chronic inflammation. Suitably, the condition relates to the kidney, liver, or lung. For example, the condition maybe inflammatory liver damage, inflammatory kidney damage or inflammatory lung damage.

Suitably, the hypoimmunogenic macrophages in the cell population may be formulated into a pharmaceutical composition. Suitably the composition is suitable for administration to a subject. Suitably the composition is a liquid. Suitably the composition is an infusible liquid.

Suitably, delivery to a subject is by systemic administration, suitably by systemic injection.

Suitably the cell population of hypoimmunogenic macrophages are for administration to a subject by any route. Suitably the cell population of hypoimmunogenic macrophages are for administration to a subject by infusion. Suitably the cell population of hypoimmunogenic macrophages are for administration to a subject parenterally, suitably intravenously. Suitably the cell population of hypoimmunogenic macrophages are for administration to a subject by injection or infusion. Suitably the cell population of hypoimmunogenic macrophages are for administration to a subject intravenously by infusion.

Suitably the cell population of macrophages are for administration to a subject at a dose of about 10⁵ to 10⁹ cells, suitably about 10⁶ to 10⁸ cells, suitably about 10⁷ cells. Suitably an appropriate dose may be determined by the medical professional based on weight, sex and age of the subject for example.

Suitably the cell population of macrophages are for administration to a subject in a single dose or multiple doses. Suitably doses may be given at intervals. Suitably doses may be given three times a day, once a day, once every two days, once every four days, once a week, once every two weeks, once every month, once every two months, once every few months, once a year, for example. An advantage of the present invention is that multiple doses may be given.

The cell population of macrophages may be for administration to a subject at an interval of once per month. The macrophages may be for administration to a subject in three doses at an interval of once per month. The macrophages may be for administration to a subject in three doses of 10⁹ cells at an interval of once per month.

The cell population of macrophages of the invention are for use as a medicament. The cell population of macrophages of the invention are for use in the treatment of a disease.

Suitably the cell population of macrophages of the invention are for use in the treatment of a chronic disease, preferably a chronic inflammatory disease.

Suitably the cell population of macrophages of the invention are for use in the treatment of chronic disease by improving regeneration, suitably by reducing expression of anti-regenerative cytokines.

Suitably the cell population of macrophages of the invention are for use in the treatment of a chronic disease by reducing fibrosis and/or reducing inflammation, suitably by reducing expression of pro-fibrotic and/or pro-inflammatory cytokines.

Suitably therefore the cell population of macrophages may be for use in the treatment of fibrotic or inflammatory diseases, suitably for use in the treatment of diseases which involve fibrosis and/or inflammation. Suitably the cell population of macrophages may be for use in the treatment of a disease by reducing fibrosis and/or by reducing inflammation.

### Definitions

A ‘macrophage’ as used herein refers to a phagocytic cell which is responsible for detecting, engulfing and destroying pathogens and apoptotic cells. A macrophage may be produced through the differentiation of any suitable precursor cell, including monocytes and PSCs. For example, an PSC cell has the ability to differentiate into any terminally or non-terminally differentiated cells. The present invention describes a stepwise method of differentiating PSCs to macrophages without generating an intermediate cell such as hematopoietic stem cell. Macrophages are usually characterised by the expression of the following markers: CD14, CD45, 25F9, CD206, CD163. Preferably, the macrophages obtained here express two or more of CD14, CD45, 25F9 or CD206.

‘Unpolarised macrophage’ as used in the present invention refers to a mature macrophage which has not received any further stimulation to induce specific functional capacity, unpolarised macrophages may also refer to naïve or non-activated macrophages.

‘functional macrophage’ as used in the present invention refers to a mature macrophage, which may be polarised or unpolarised, displaying the expected characteristics of macrophages. In particular, the functional macrophage may be capable of phagocytosis, being polarised, and expressing the surface markers and cytokines associated with its polarisation state.

‘polarised macrophage’ as used in the present invention refers to a macrophage which has received environmental stimulus to become activated into a particular phenotype such as the pro-inflammatory or pre-regenerative phenotype.

An ‘pro-inflammatory polarising factor’ as used in the present invention refers to a factor which stimulates an unpolarised macrophage into an pro-inflammatory phenotype, and may refer to one or more of: GM-CSF, IFNγ, and TLR agonists, such as LPS, for example.

An ‘pro-regenerative polarising factor’ as used in the present invention refers to a factor which stimulates an unpolarised macrophage into an pro-regenerative phenotype, and may refer to one or more of: IL10, IL4, IL13, and poly(I:C), for example.

As used herein, the term “hypoimmunogenic” means that a cell is less immunogenic i.e. less prone to provoke an immune response compared to an unaltered cell. The immunogenicity of a hypoimmunogenic cell could be reduced by 5%, 10%, 20%, 30%, 40%, 50% 60%, 70%, 80%, 90% or 100% compared to an unaltered cell. The immunogenicity of cells may be measured, for example, by the production of antibodies directed against antigens present in the cells upon administration of the cells to a host. For example, immunogenicity may be measured via the generation of anti-HLA antibodies, or antibodies directed against minor histocompatibility antigens expressed by the hypoimmunogenic functional macrophages according to the present invention. Relative reduction in immunogenicity may be determined by comparing the quantity of antibody produced by the host upon administration of the hypoimmunogenic functional macrophages of the present invention compared to functional, non-hypoimmunogenic cells which express HLA molecules. As an example, cells that are essentially devoid of functional HLA I and HLA II complexes on their surface are hypoimmunogenic. Accordingly, any aspect of the invention may relate to a method for producing macrophages that are essentially devoid of functional HLA I and HLA II complexes on their surface.

As a result of HLA-I and HLA-II loss, hypoimmunogenic cells evade immune recognition by host T cells, i.e., they do not elicit allogeneic-T cell proliferation, activation and effector or cytotoxic T cell responses towards the hypoimmunogenic cell.

As used herein, “an unaltered cell” is a cell that has not undergone any genetic modification.

‘mature macrophage’ refers to a macrophage which expresses mature cell surface markers, preferably CCR2−, CD14+ and 25F9+. ‘GMP-compliant’ as used herein means that the method complies with Good Manufacturing Practice principles and may be used interchangeably with ‘GMP-compatible’ and ‘GMP-graded’. By way of example a GMP-compliant medium has to be serum-free, antibiotic-free, animal substance free and xenoprotein-free. The WHO provides guidance on what is required for good manufacturing practice: “Chapter 1: WHO good manufacturing practices: Main principles for pharmaceutical products”. Quality Assurance of Pharmaceuticals: A compendium of guidelines and related materials—Good manufacturing practices and inspection. 2 (2nd updated ed.). WHO Press. pp. 17-18. ISBN 9789241547086.

It is to be noted that the term “a” or “an” entity refers to one or more of that entity.

‘about’ means+/−10% of the value given, +/−9%, +/−8%, +/−7%, +/−6%, +/−5%, +/−4%, +/−3%, +/−2%, +/−1%, unless otherwise stated.

The term “knock-out” means that a gene has been silenced or inactivated such that the gene no longer encodes a functional protein. The term “knock-down” means that the function of a gene has been reduced or interfered such that the gene encodes reduced levels of the protein or the gene encodes a protein with reduced function.

As used herein, “iMACs” or “induced macrophages” are macrophages that are derived from iPSCs.

‘treatment’ as used in the present invention means an intervention in a physiological condition which prevents, reduces, or removes the clinical symptoms associated with a given physiological condition in a subject.

By ‘subject’ or ‘individual’ or ‘animal’ or ‘patient’ is meant any subject, particularly a mammalian subject, for whom diagnosis, prognosis, or therapy is desired, except where the subject is defined as a ‘healthy subject’. Mammalian subjects include humans; domestic animals; farm animals; such as dogs, cats, guinea pigs, rabbits, rats, mice, horses, cattle, cows, and so on.

Small interfering RNA (siRNA), sometimes known as short interfering RNA or silencing RNA, is a class of double-stranded RNA at first non-coding RNA molecules, typically 20-24 (normally 21) base pairs in length, and operating within the RNA interference (RNAi) pathway.

All documents including patent applications mentioned herein are incorporated by reference.

The invention will now be described with reference to the following non-limiting Examples.

### EXAMPLES

### Example 1—Differentiation of Hypoimmunogenic iPSCs to iMACs Progenitor

iPSCs were cultured using cell therapy grade reagents, mTeSR Plus medium (StemCell Technologies Cat. No. 100-0276) and human recombinant laminin 521 (Biolamina Cat. No. LN521-05) at 5 μg/mL in 1× DPBS (Gibco Cat. No. 14040133) (with Ca2+/Mg2+) were used. Media was changed every 24 to 48 hrs. Cells were split once cell colonies reached 80% confluency using ReLeSR (StemCell Technologies Cat. No. 05872).

PSC characterisation was done regularly to ensure pluripotency is maintained under culture. For extracellular markers, PSCs were resuspended at a concentration of 1×10⁶/ml in PBS+0.5 mM EDTA (Life Technologies)+FcR Block 1:100 (Miltenyi). The following protocol was used. Dispense 100 μL of cells into low adherence, round bottomed 96 well plates. Incubate cells for 5 minutes, then add appropriate antibodies (TRA-1-60, SSEA-1 and SSEA-4 see Table 1) to appropriate test wells and leave for 20 min at 4 C. Wash cells with PBS+0.5 mM EDTA and spin at 300 g for 5 min. Flip off the supernatants and resuspend cells in PBS+0.5 mM EDTA+1:1000 DRAQ7. Incubate for 5 min at 4 C. Wash as before, then resuspend in 100 ul of PBS+0.5 mM EDTA+0.1% human serum. Acquire 50 paL of cells on the Novocyte3000 or Novocyte Quanteon (Agilent). For intracellular markers, PSC were resuspended at a concentration of 1×10⁶/ml in PBS+0.5 mM EDTA (Life Technologies)+Zombie NIR (Biolegend, prepared as per manufacturer's instructions), prepare similar suspension without Zombie NIR for unstained control. Dispense 100 μL of cells into low adherence, round bottomed 96 well plates. Incubate cells for 15 minutes at room temperature (RT) in the dark. Wash by spinning at 300 g for 5 mins and resuspend in 100 μL of PBS+0.5 mM EDTA. Add 100 μL of 4% paraformaldehyde (PFA) onto the wells and fix the cells for 15 min at 4 degrees Celsius in the dark. Wash by spinning at 300 g for 5 mins and resuspend in 100 μL of PBST supplemented with FcR Block 1:100 for 15-30 min at RT in the dark on a plate rocker/shaker. Add the appropriate antibodies (OCT3/4, Nanog, see Table 1) to appropriate test wells and leave for 20 min at 4 C. Wash as before, then resuspend in 100 ul of PBS+0.5 mM EDTA+0.1% human serum. Acquire 50 μL of cells on the Novocyte3000 or Novocyte Quanteon (Agilent).

TABLE 1
Antibodies used for flow cytometry labelling of iPSC.

| | | | | Catalog |
|---|---|---|---|---|
| ANTIGEN | FLUOROPHORE | CLONE | SUPPLIER | No. |
| TRA-1-60 | Per/Cyanine 5.5 | TRA-1- | BioLegend | 330618 |
| | | 60R | | |
| SSEA-1 | Pacific Blue | MMA | BioLegend | 394704 |
| SSEA-4 | APC | MC-813- | BioLegend | 330418 |
| | | 70 | | |
| OCT3/4 | APC | REA662 | Miltenyibiotec | 130-123- |
| | | | | 257 |
| NANOG | PE | REA314 | Miltenyibiotec | 130-117- |
| | | | | 337 |

The iMACs differentiation protocol used consists of a stage wise differentiation where embryonic bodies (EBs) are generated from PSC using low attachment plates or wells as per van Wilgenburg et al., 2013.³¹

EBs are then transferred into culture flasks for iMACs progenitor generation. iMACs progenitors are generated in a continuous manner for up to two months, weekly harvests being performed and the iMACs progenitors are plated for iMACs maturation.

PSC monolayer must be 60-70% confluent on the day of seeding for EB formation. A single cell suspension is generated using ReLesR. EBs can be generated using two different methods AggreWell™800 (StemCell Technologies) or ultra-low attachment (ULA) 96 well plates. EBs were generated by dispensing a single cell suspension in MTeSR plus+BMP4, SCF, VEGF and Rock Inhibitor (Y-27632). The required volume of cells is seeded into the EB generation platform being used. Gently spin down the plate (ULA 96 well plate or AggreWell™800™ 800) at 100G for 3 minutes. Incubate at 37° C. 5% CO₂ for 7 days. For AggreWell™800 culture, perform daily half medium change with MTeSR plus+BMP4, SCF and VEGF. For 96 ULA well plate, perform half medium change every 48 h with MTeSR plus+BMP4, SCF and VEGF.

### Example 2—iMACs Progenitor Generation and Maturation to iMACs

Following EBs generation, the EBs are gently collected and seeded in low adherence flasks cultured on vertical in presence of M-CSF and IL-3. These culture systems are then referred to as ‘iMACs Factories’. They are incubated at 37° C. for up to 8 weeks with weekly harvests. To harvest the cells, remove the supernatant to collect iMACs progenitors without removing EBs by allowing for the EBs to sink under gravity force prior collection.

iMACs progenitors are then seeded and cultured in presence of M-CSF for a sufficient period of time for further maturation.

iMACs characterisation was done at both progenitor and matured stages. iMACs were resuspended at a concentration of 1×10⁶/ml in PBS+0.5 mM EDTA (Life Technologies)+FcR Block 1:100 (Miltenyi). Dispense 100 ul of cells into low adherence, round bottomed 96 well plates. Incubate cells for 5 minutes, then add appropriate antibodies (see Table 2) to appropriate test wells and leave for 20 min at 4 C. Wash cells with PBS+0.5 mM EDTA and spin at 300 g for 5 min. Flip off the supernatants and resuspend cells in PBS+0.5 mM EDTA+1:1000 DRAQ7. Incubate for 5 min at 4 C. Wash as before, then resuspend in 100 ul of PBS+0.5 mM EDTA+0.1% human serum. Acquire 50 ul of cells on the Novocyte3000 or Novocyte Quanteon (Agilent).

TABLE 2
Antibodies used for flow cytometry labelling of iMACs.

| | | | | Catalog |
|---|---|---|---|---|
| ANTIGEN | FLUOROPHORE | CLONE | SUPPLIER | no. |
| CD45 | VB | 5B1 | Miltenyi | 130-092-880 |
| | | | Biotec | |
| CD14 | VB/PE | TUK4 | Miltenyi | 130-091-242/ |
| | | | Biotec | 130-113-152 |
| CD206 | FITC | — | Miltenyi | 130-095-131 |
| | | | Biotec | |
| 25F9 | APC | 25F9 | eBioscience | 50-0115-42 |

### Example 3—MAC Polarisation

iMAC polarisation was assessed by cell surface marker expression and cytokine secretion analysis (MSD V-Plex). For M0 controls, mature iMACs were resuspended at a density of 3×10\hat{ }6/ml in TexMACS+100 ng/mL M-CSF. For M2 polarisation, mature iMACs were resuspended at a density of 3×10\hat{ }6/ml in TexMACS+100 ng/mL M-CSF+50 ng/mL IL-10 or in TexMACS+100 ng/mL M-CSF 20 ng/mL of IL4 & IL13. For M1 polarisation, mature iMACs were resuspended at a density of 3×10\hat{ }6/ml in TexMACS+100 ng/mL LPS+50 ng/mL IFN-γ. iMACs were then seeded in a 96-well clear, flatbottom plate and incubated for 24 hours at 37 degrees Celsius, 5% CO₂. After polarisation, cell supernatant was collected for MSD assessment and cells were harvested for flow cytometry assessment.

TABLE 3
Antibodies used for flow cytometry assessment
of macrophage polarisation

| | | | | Catalog |
|---|---|---|---|---|
| ANTIGEN | FLUOROPHORE | CLONE | SUPPLIER | no. |
| CD14 | VioBlue | TUK4 | Miltenyi | 130-113-152 |
| CD45 | PerCP | TD1 | Biolegend | 368505 |
| CD206 | BV711 | 15-2 | Biolegend | 321136 |
| 25F9 | eF660 | eBio25F9 | invitrogen | 50-0115-42 |
| CD163 | PeCy7 | GHI61.1 | Miltenyi | 130-101-484 |
| CD86 | PE | BU63 | Biolegend | 374206 |

Cytokines in cell culture supernatants were analysed using a V-PLEX Human Biomarker 10-Plex kit on a MESO Quickplex SQ 120 according to the manufacturers' instructions (Meso Scale Discovery). Conditioned medium from macrophages (human or IPSC-derived) were accumulated for 24 hours and harvested from cultures. 25 μL of supernatants were tested. Results are in pg/mL. Values are adjusted taking into consideration the dilution at the time of testing. All data shown represent the secretion in a 24 h period. Data reported are net concentration, as calculated by subtracting the amount of the given cytokine in culture medium alone (TexMACS) to the amount of cytokine detected in the cell culture supernatants.

### Example 4—Functional Characterisation of iMACs

iMAC phagocytic potential was measured via imaging, or flow cytometry. Imaging analysis was utilised to measure the kinetics of iMAC phagocytosis, in real time, whereas flow analysis was utilised for end-point measurement.

iMACs were prepared for imaging analysis as follows: iMACs were plated at a density of 150,000 cells/well in a 96-well clear bottom imaging plate (Grenier) and matured for 5 days on TexMACS+100 ng/mL M-CSF. Supernatant was removed and cells were stained for 30 mins with 100 ul PBS+NucBlue (ThermoFisher)+5 μg/ml Cellmask Deep red plasma membrane stain (Invitrogen) at 37 degrees Celsius, 5% CO₂. Cells were washed three times with 100 μl PBS. 50 μl of PBS was added to cells for TO analysis on the Opera Phenix High Content Screening System. 50 μl of 0.2 mg/ml pHrodo Red Zymosan beads (Life Technologies) were added to cells after T0. A series of images were acquired over a period of 96 minutes to monitor phagocytosis. Images were analysed with Columbus data imaging software and Tibco Spotfire data analysis system. Graphs plotted using GraphPad Prism 9.2.0.

iMACs were prepared for flow cytometric analysis as follows: matured iMACs were resuspended at a concentration of 2×106/ml in PBS+0.5 mM EDTA (Life Technologies). Dispense 50 μL of the cell suspension into low adherence, round bottomed 96 well plates. Add 50 μL of resuspended pHrodo Beads (prepared as per manufacturer's instructions) to the test wells. Incubate cells for 2 hours at 37° C. 5% CO2. At the end of the 1 hr incubation, spin the plate 300 xg, 4° C., 5 min. Eliminate supernatants and resuspend the pellet with 100 μL of 1:100 FcR block PEA solution/well. Incubate for 15 min at 4° C. in dark then add appropriate antibodies (see Table 3) to appropriate test wells and incubate for 20 min at 4 C. Wash cells with PBS+0.5 mM EDTA and spin at 300 g for 5 min. Flip off the supernatants and resuspend cells in PBS+0.5 mM EDTA+1:1000 DRAQ7. Incubate for 5 min at 4 C. Wash as before, then resuspend in 100 ul of PBS+0.5 mM EDTA+0.1% human serum. Acquire 50 ul of cells on the Novocyte3000 or Novocyte Quanteon (Agilent). Flow cytometry analysis was conducted on NovoExpress software and the following gating strategy was utilised to identify actively phagocytosing iMACs: “Cell gate” to exclude debris, “singlet gate” to exclude cell doublets, “live gate” to exclude dead cells, “CD14+ gate” to identify iMACS and “phRodo+ve gate” to measure the percentage of phagocytosing iMACs.

TABLE 4
Antibodies used for flow cytometry phagocytosis.

| | | | | Catalog |
|---|---|---|---|---|
| ANTIGEN | FLUOROPHORE | CLONE | SUPPLIER | no. |
| pHrodo Red E. | — | — | Invitrogen | P35361 |
| Coli | | | | |
| BioParticles | | | | |
| Conjugate for | | | | |
| Phagocytosis | | | | |
| CD14 | VB | TUK4 | Miltenyi | 130-113-152 |
| | | | Biotec | |

### Example 5—iPSC without HLA-I and HLA-II Express Pluripotency Markers

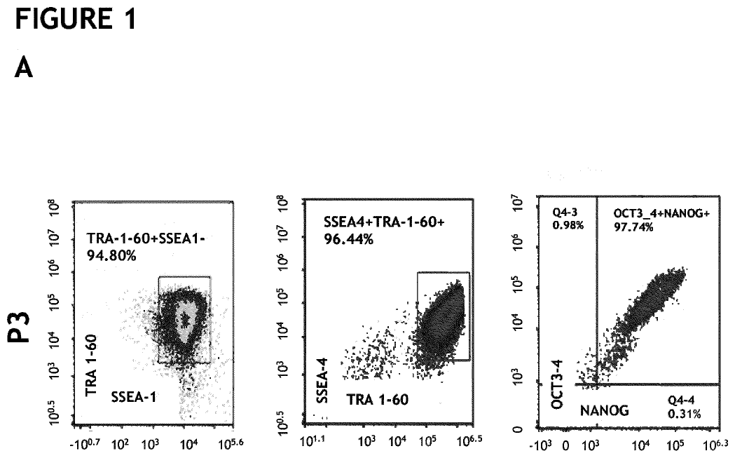

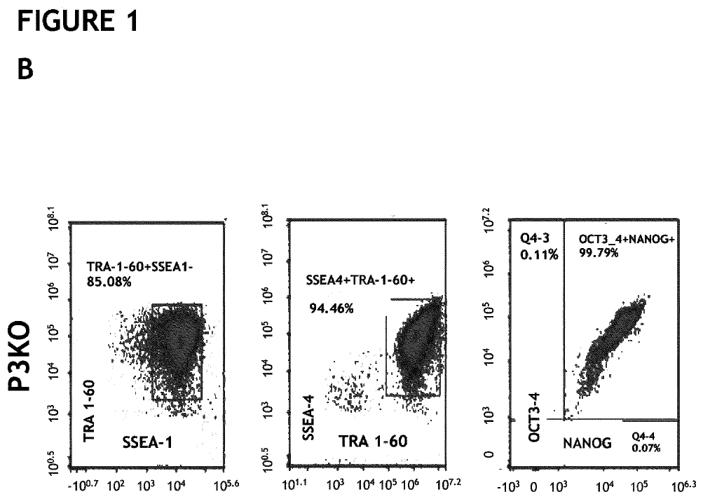

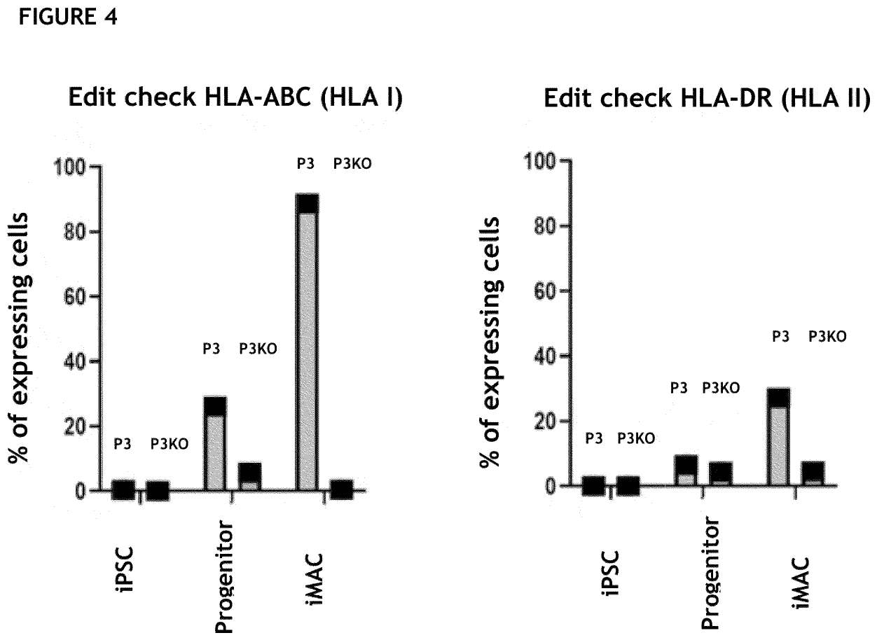

iPSCs were cultured as described in Example 1 and the expression of different pluripotency markers was assessed by flow cytometry. The same pluripotency markers were compared in two cell lines, termed “P3” and “P3KO”. The P3 cells are iPSCs which have been generated essentially as described in (Baghbaderani, Syama et al. 2016). Briefly, cells were reprogrammed using umbilical Cord Blood (hUCB) CD34+ cells and episomal plasmids encoding Oct4, Sox2, Klf4, c-Myc and Lin28. The P3KO cells are similar to the P3 cells but contain a knockout in the B2M and CIITA genes which results in no expression of HLA I and HLA II on the surface of the iPSC cells and cells differentiated from them. P3KO cells were generated using a nuclease-based approach (CRISPR/Cas9) inducing indels in both genes, B2M and CIITA. The difference in HLA expression between P3 and P3KO is demonstrated in FIG. 4. Both P3 and P3KO displayed similar levels of expression for all pluripotency markers. Absence of HLA I and II in P3KO PSC line does not affect PSC pluripotency markers expression (FIG. 1). We tested the standard pluripotency markers, described in the original iPSC generation publication {Baghbaderani, 2016 #2}

### Example 6—Successful Differentiation of iMACs Using GMP Compatible Reagents

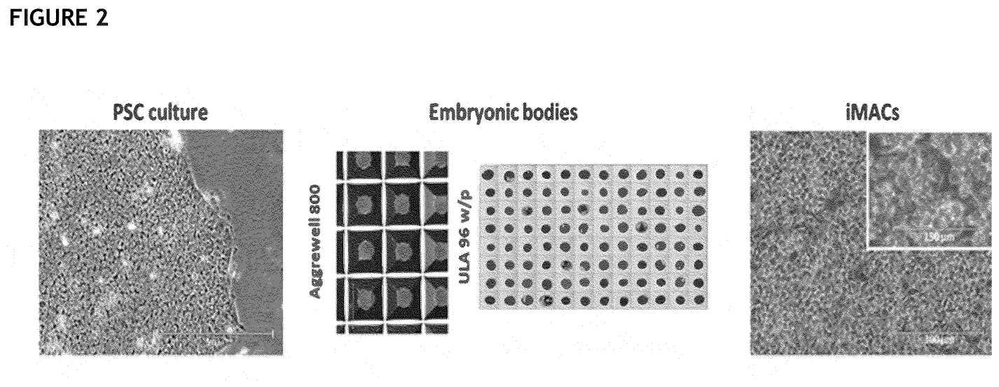

iMACs were differentiated using a stepwise differentiation where PSC were differentiated into embryonic bodies (EBs) as described in Example 1. Next, EBs were transferred into a T-75 flask used vertically and the iMACs progenitor factory stage was initiated as in Example 2. This stage can be maintained up to two months where iMACs progenitors are generated constantly. Every week, medium is collected to harvest iMACs progenitors for further maturation (FIG. 2).

iMACs progenitors were collected weekly for characterisation by flow cytometry. Successful criteria for iMACs differentiation was achieved when iMACs progenitors expressed >80% of positive of macrophage markers (CD45, CD14, CD206 and 25F9) Once that threshold was met, the differentiation was considered successful and the weekly iMACs progenitors harvested from that point were used for maturation and characterisation.

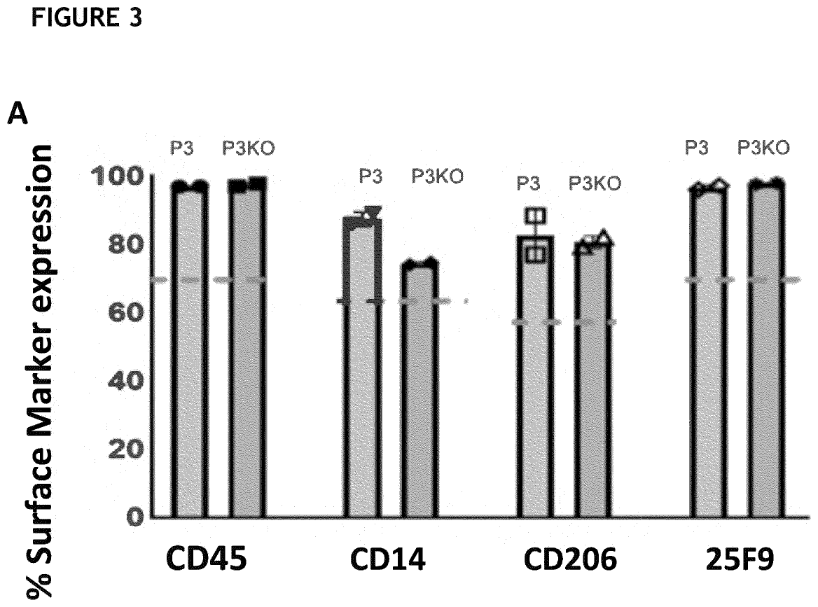

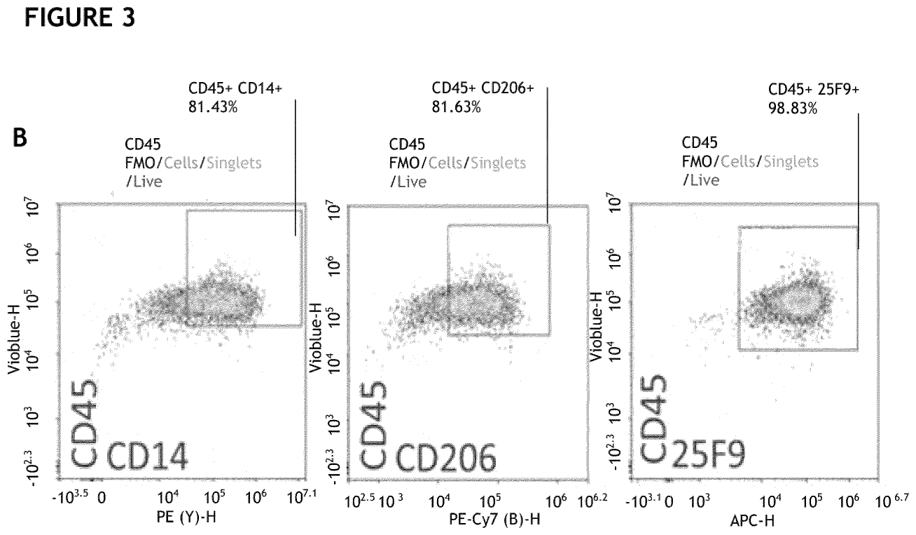

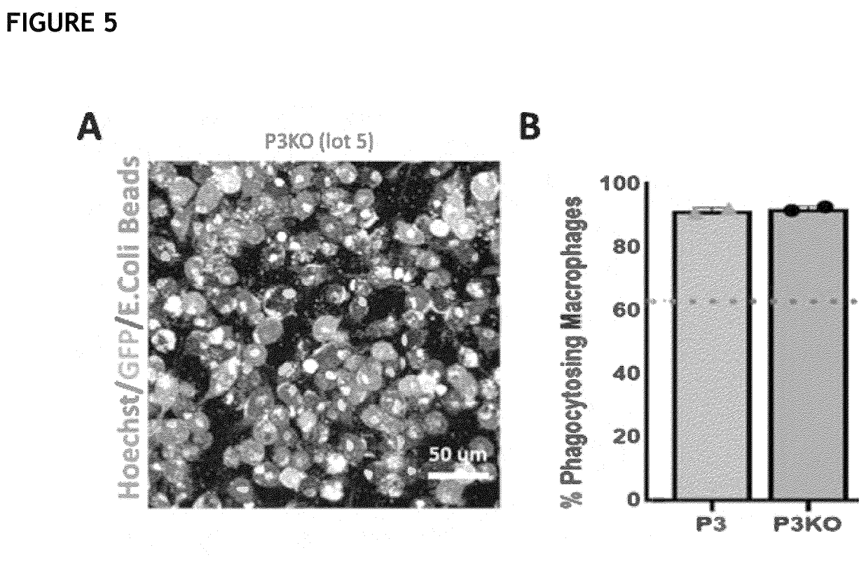

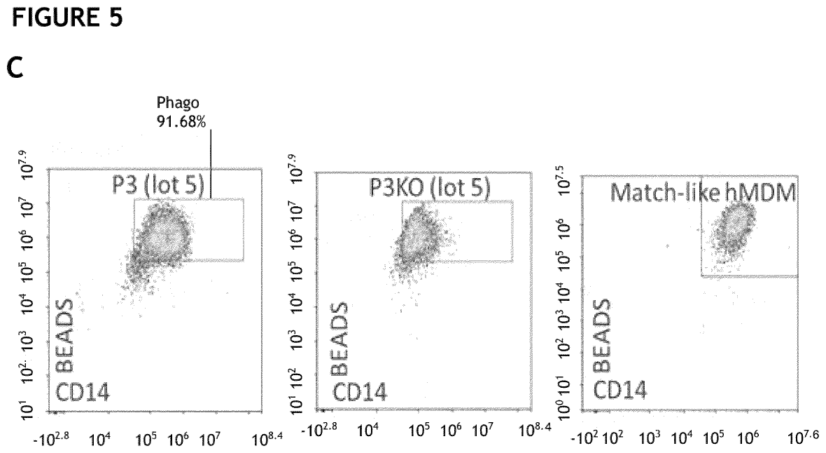

iMACs maturation was performed for a sufficient period of time until matured iMACs expressed >70% of positive cells of macrophage markers (CD45, CD14, CD206 and 25F9; FIG. 3) and displayed phagocytic capabilities >60% positive phagocytic cells (FIG. 5). Phagocytic cells are identified by being positive for both CD14 and pHrodo. Therefore, the iMACS were deemed mature when >60% of cells in the sample were positive for both CD14 and pHrodo.

### Example 7—Absence of HLA I and II Expression

HLA I and 11 expression in both P3 and P3KO was assessed to ensure P3KO does not express HLA I and 11 in any stage of the differentiation. For this, PSC, iMACs progenitors and matured iMACs HLA I and HLA II expression was analysed by flow cytometry (FIG. 4) using the antibodies noted in Table 5 below. No expression of HLA I or HLA II was observed in P3KO cells at any point of the differentiation. Table 5. Antibodies used for flow cytometry HLA expression analysis.

| | | | | Catalog |
|---|---|---|---|---|
| ANTIGEN | FLUOROPHORE | CLONE | SUPPLIER | no. |
| HLA-DR/DP/DQ | PeCy7 | Tu39 | Biolegend | 361708 |
| HLA-ABC | PE | W6/32 | Biolegend | 311405 |

### Example 8—Matured iMACs are Phagocytic and Responsive to Polarisation

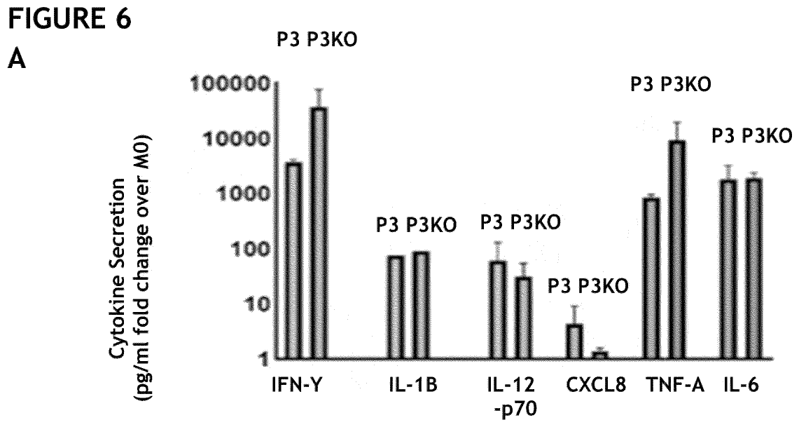

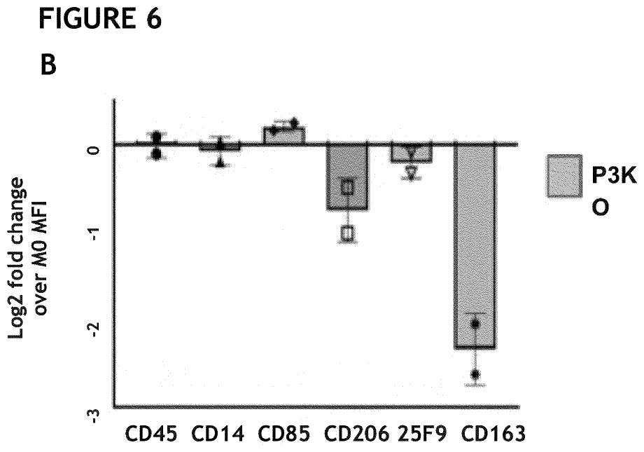

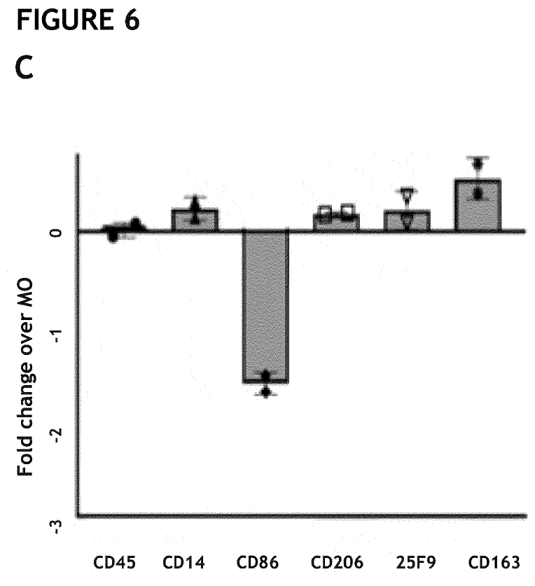

To measure mature iMAC function, iMACs were assessed for their ability to phagocytose (FIG. 5) and polarise towards M1 and M2 phenotypes (FIG. 6). Ability to phagocytose was assessed according to the method described in Example 4, and polarisation was assessed according to the method described in Example 3.

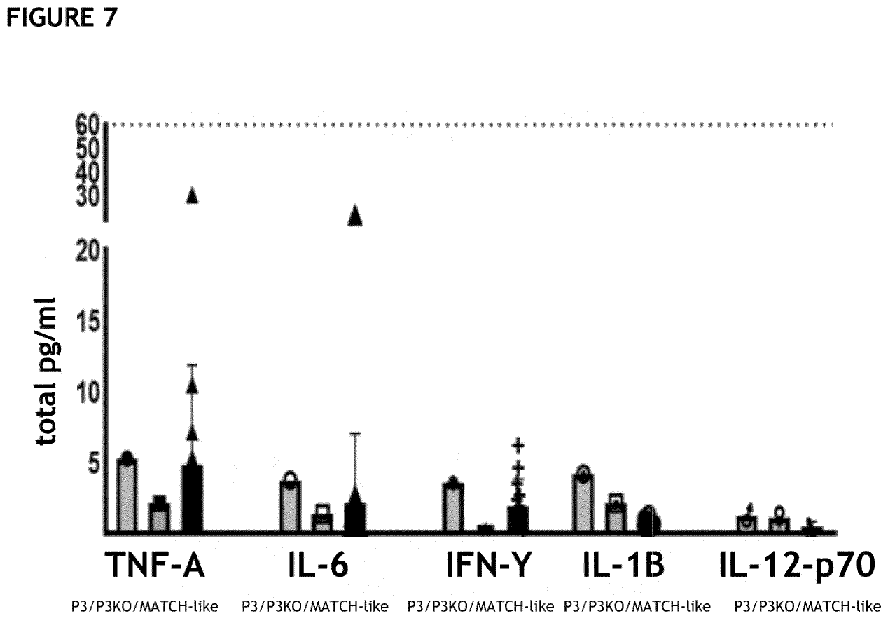

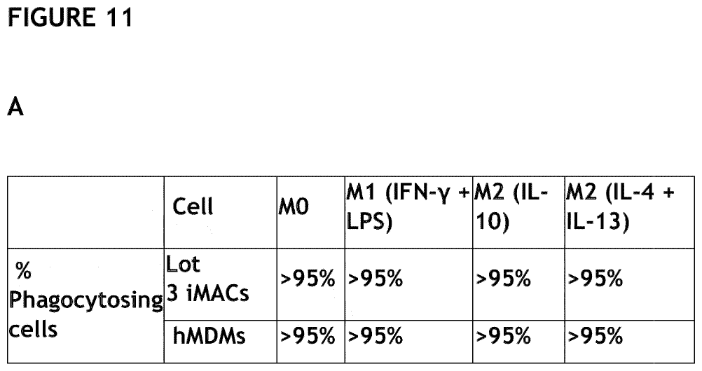

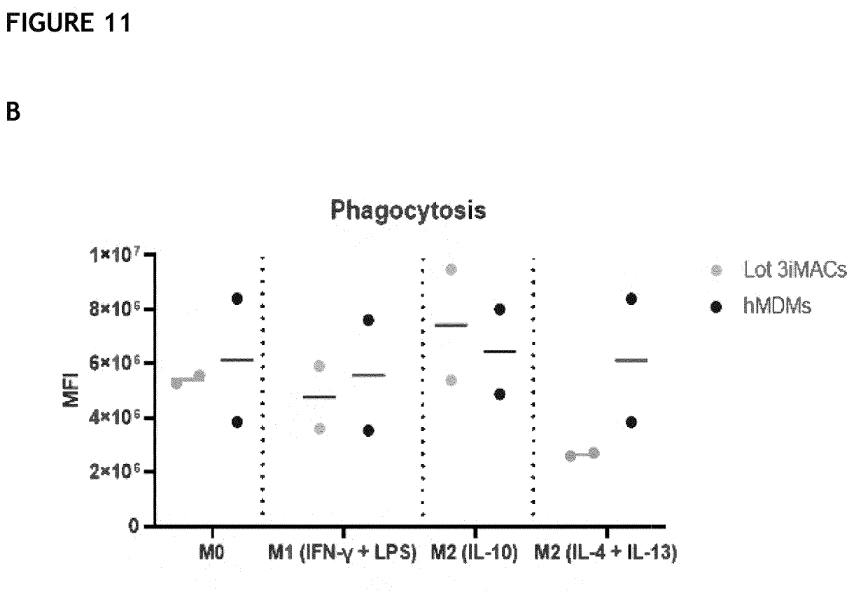

>90% of mature iMACs generated from P3 or P3KO cells could phagocytose as measure by e. coli-bead+ve CD14+ve iMACs (FIG. 5). The phagocytic capacity of non-polarised P3 and P3KO iMACs phagocytic capacity was measured via imaging (FIG. 5A) and flow cytometry (FIG. 5B&C). >91% of P3 and 92% of P3KO iMACS (FIG. 5B). As can be seen in FIG. 5C, the percentage of CD14+ mature non-polarised human monocyte derived macrophages (termed Match-like hMDM in FIG. 5C). The comparable phagocytosis of iMACS and hMDMs in various polarisation stages is further shown in FIG. 11. M1 iMAC polarisation was confirmed by an increase in the secretion of IL-6 and TNF-A, compared to M0 iMACs, both in iMACs derived from P3 and P3KO cells (FIG. 6A). Successful M1 polarisation in iMACs derived from P3KO cells was further demonstrated by upregulation of the M1 marker CD86 and down-regulation of the M2 markers CD206, 25F9 and CD163 as compared to non-polarised iMACs derived from P3KO cells (FIG. 6B). Successful M2 polarisation in iMACs derived from the P3KO iPSCs was confirmed by an increase in the surface marker expression of the M2 markers CD206, 25F9 and CD163 as compared to non-polarised iMACs (FIG. 6C). Using our protocol, matured non-polarised iMACs show a non-inflammatory profile, similar to PBMC-derived macrophages (“MATCH-like”) (FIG. 7).

### Example 9—Polarised and Unpolarised HLA-Deficient iMACs Exhibit Reduced IL-10 Signalling Receptor (IL-10RA) Transcriptional Expression Compared to hMDMs

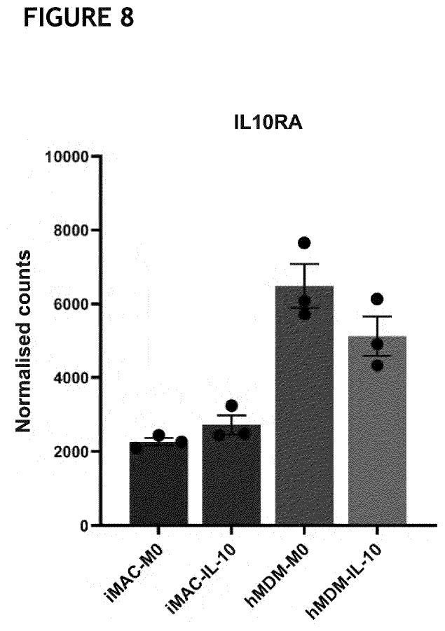

The inventors sought to determine whether HLA-deficient iMACs (derived from the P3KO iPSC cell line) had altered expression of IL-10RA. In particular, the expression of IL-10RA was measured in unpolarised (M0) iMACs and hMDMs, and iMACs and hMDMs polarised with IL-10 (IL-10), by the same protocol described in Example 3, except that polarisation with IL-10 was performed using 10 ng/mL IL-10. IL-10RA mRNA was detected and quantified using the immunology codeset from the NanoString nCounter system. Data import, quality control and normalisation of expression values were conducted using the nSolver version 4.0 (Nanostring Technologies, Inc.). Background subtraction from raw transcript counts was performed using a threshold count value and gene expression normalisation was performed by housekeeping-normalisation by dividing the geometric mean of 15 housekeeping genes.). iMACs were found to exhibit reduced IL-10RA expression compared to hMDMs which express functional HLA complexes on their surface. iMACs had reduced expression of IL-10RA compared to hMDMs regardless of polarisation state (FIG. 8).

Interestingly, publicly available databases (e.g. as described in Zhang et al., 2015, Circ Res. 117(1):17-28 and Rajab et al., 2021, Stem Cell Reports, 8; 16(6):1629-1643) suggest that the expression level of IL10-RA on iPSC-derived macrophages (iMACs) which are not HLA-deficient is similar or higher than that found in PBMCs or hMDMs (data not shown).

### Example 10—Mature HLA-Deficient iMACs Exhibit Comparable Surface Marker Profile to hMDMs in Different Polarisation States

The inventors aimed to characterise the surface marker expression profile of mature HLA-deficient iMACs derived from the P3KO cell line. This experiment had two goals. The first goal was to assess the surface marker expression of iMACs and how the expression of the different markers changes with polarisation. This would demonstrate whether iMACs behave like macrophages following polarisation and to assess how resting/M0 iMACs compare to iMACs polarised towards a pro-inflammatory phenotype [M1 (IFN-γ+LPS)] and to an anti-inflammatory phenotype [M2 (IL-10) and M2 (II-4+IL-13)], with the polarisation performed as described in Example 3, except that polarisation with IL-10 was performed using 10 ng/mL IL-10. The second goal was to compare iMACs and hMDMs in the levels of expression and pattern of expression of the different markers in the different polarisation states.

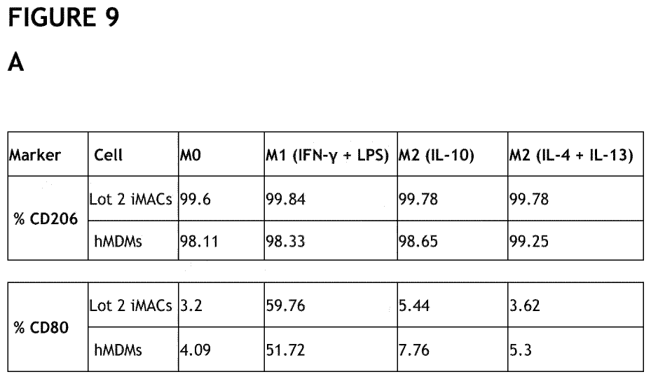

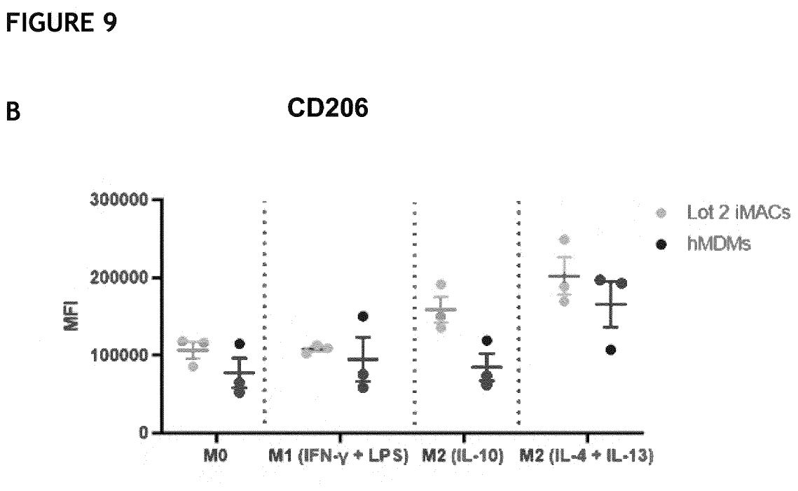

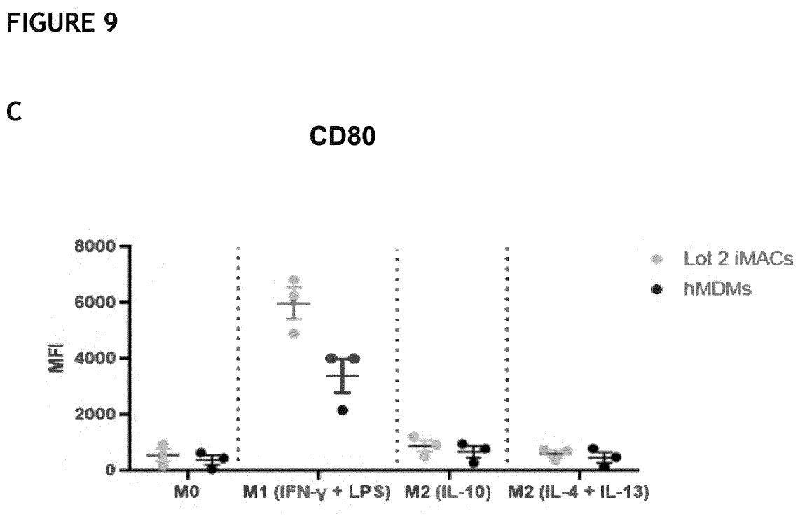

CD206 expression in macrophages is associated with an anti-inflammatory phenotype. One of the success parameters set for this experiment was to observe an increase in CD206 expression in M2 (IL-10) and M2 (IL-4+IL-13) compared to M0 and M1 (IFN-γ+LPS) polarised macrophages. Therefore, in addition to assessing the frequency of live iMACs and hMDMs expressing CD206, we assessed the median fluorescence intensity (MFI) to evaluate the expression levels of these markers in the different polarisation states (FIG. 9B). No differences in CD206 MFI were observed between M0 iMACs and M0 hMDMs. Moreover, CD206 expression in M1 (IFN-γ+LPS) polarised iMACs and hMDMs was similar to that in corresponding M0 cells. CD206 was upregulated in M2 (IL-10) and M2 (IL-4+IL-13) iMACs compared to M0 and M1 (IFN-γ+LPS) polarised iMACs. The upregulation of CD206 was not observed in M2 (IL-10) polarised hMDMs whereas it was slightly upregulated in M2 (IL-4+IL-13) compared to M0 and M1 (IFN-γ+LPS) polarised hMDMs. As a result, M2 (IL-10) polarised iMACs expressed higher levels of CD206 compared to M2 (IL-10) polarised hMDMs, whereas similar levels of expression were observed between M2 (IL-4+IL-13) iMACs and M2 (IL-4+IL-13) hMDMs.

Expression of CD80 in M0, M2 (IL-10) and M2 (IL-4+IL-13) polarised cells was similar between iMACs and hMDMs. Expression of CD80 was higher in M1 (IFN-γ+LPS) polarised iMACs compared to M1 (IFN-γ+LPS) polarised hMDMs (FIG. 9C).

Accordingly, it may be seen that iMACs may be polarised as effectively as hMDMs, and indeed, may polarise more efficiently in response to a given polarisation stimulus. This is particularly surprising, in light of the reduced expression of the IL-10RA shown on the cell surface, which could suggest that IL-10 would be less effective in promoting M2 polarisation.

### Example 11—Cytokine Secretion of HLA-Deficient iMACs Compared to hMDMS in Various Polarisation States

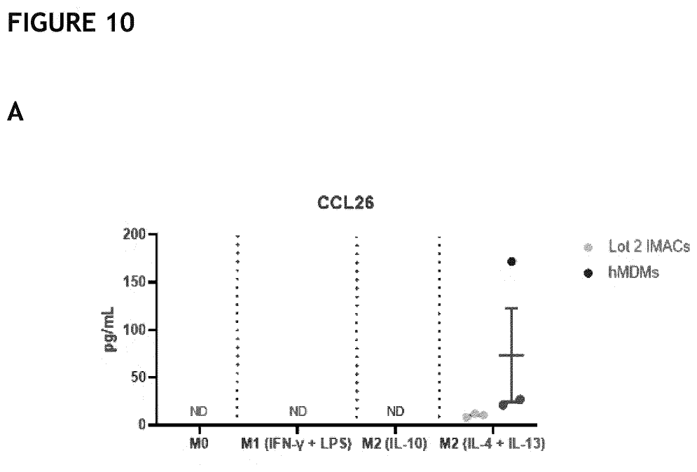

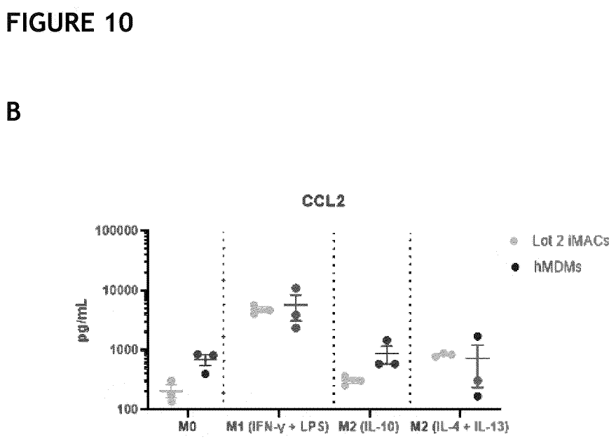

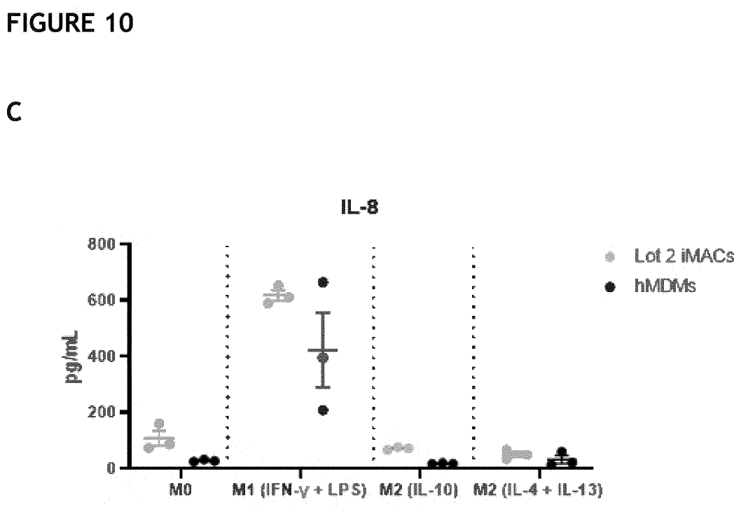

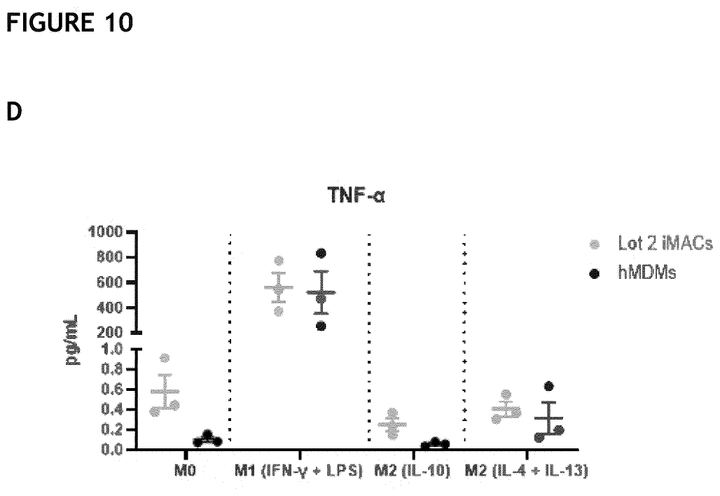

To further characterise the function of HLA-deficient iMACs (derived from the P3KO iPSC cell line), the secreted levels of both anti-inflammatory cytokines (CCL26, as shown in FIG. 10A) and inflammatory cytokines (CCL2, IL-8, TNF-α, as shown in FIGS. 10B, 10C and 10D, respectively) were measured as described in Example 3. In general, iMACs secreted comparable levels of the tested cytokines to hMDM counterparts of the same polarisation state. Thus, the data in FIG. 10 shows that hMDMS and iMACs demonstrate similar properties in terms of cytokine secretion and show that iMACs may be successfully polarised similarly to hMDMs.

### Example 12—Phagocytic Activity in Polarised and Unpolarised HLA-Deficient iMACs

The inventors then aimed to characterise the functionality of HLA-deficient iMACs (derived from the P3KO iPSC cell line) by assessing their phagocytic capacity. This experiment had two goals. The first goal was to assess the phagocytic capacity of HLA-deficient iMACs developed from HLA-deficient iPSCs and to assess how it changes in the different polarisation conditions. The second goal was to compare the phagocytic capacity of HLA-deficient iMACs to that of hMDMs in different polarisation states. The phagocytic capacity was assessed in M0, M1 (IFN-γ+LPS), M2 (10 ng/mL IL-10) and M2 (IL-4+IL-13) polarised iMACs and hMDMs using a phagocytosis assay by flow cytometry (essentially as described in Example 4), with the results shown in FIG. 11. The percentage of cells phagocytosing in each polarisation state is tabulated in FIG. 11A. The mean fluorescence intensity of the labelled E Coli in each polarisation state is shown in FIG. 11B.

The success parameter set for this experiment was to observe >70% phagocytic iMACs and hMDMs in the different polarisation conditions. Over 95% of iMACs and hMDMs were phagocytosing in all polarisation states. In order to assess whether the phagocytic capacity changed with the polarisation state, we assessed the MFI. Polarisation towards M1 and M2 did not change the phagocytic activity in iMACs nor hMDMs compared to the corresponding M0 cells and no differences between iMACs and hMDMs were observed in any of these polarisation conditions. On the other hand, the phagocytic capacity of iMACs was found to be reduced in M2 (IL-4+IL-13) iMACs compared to M0 iMACs, whereas no differences were observed in hMDMs from M0 to M2 (IL-4+IL-13) polarised cells. As a result, the MFI is higher in M2 (IL-4+IL-13) hMDMs compared to M2 (IL-4+IL-13) iMACs and the MFI is higher in M2 (IL-10) iMACs than M2 (IL4+IL-13) iMACs.

### CONCLUSIONS

The work presented here shows a successful generation of pro-regenerative hypoimmunogenic stem cell derived macrophages from a hypoimmunogenic PSC line with low or no expression of the highly polymorphic classical MHC-1 (HLA-A, HLA-B and HLA-C) and MHC-II complexes.

### REFERENCES

1 Williams, R. et al. Lancet 384, 1953-1997, doi:10.1016/S0140-6736(14)61838-9 (2014).
2 Pellicoro, A., Ramachandran, P., Iredale, J. P. & Fallowfield, J. A. Nat Rev Immunol 14, 181-194, doi:10.1038/nri3623 (2014).
3 Fallowfield, J. A. Clin Med (Lond) 15 Suppl 6, s83-87, doi:10.7861/clinmedicine.15-6-s83 (2015).
4 Forbes, S. J. & Newsome, P. N. Nat Rev Gastroenterol Hepatol, doi:10.1038/nrgastro.2016.97 (2016).
5 Thomas, J. A., Ramachandran, P. & Forbes, S. J. J Hepatol 68, 1090-1091, doi:10.1016/j.jhep.2017.11.043 (2018).
6 Forbes, S. J., Gupta, S. & Dhawan, A. J Hepatol 62, S157-169, doi:10.1016/j.jhep.2015.02.040 (2015).
7 Falasca, L., et al Exp Cell Res 224, 152-162, doi:10.1006/excr.1996.0123 (1996).
8 Ramachandran, P. et al. Proc Natl Acad Sci USA 109, E3186-3195, doi:10.1073/pnas.1119964109 (2012).
9 Wynn, T. A. & Barron, L. Semin Liver Dis 30, 245-257, doi:10.1055/s-0030-1255354 (2010).
10 Zigmond, E. et al. J Immunol 193, 344-353, doi:10.4049/jimmunol.1400574 (2014).
11 Campana, L., et al Nat Rev Mol Cell Biol 22, 608-624, doi:10.1038/s41580-021-00373-7 (2021).
12 Thomas, J. A. et al. Hepatology 53, 2003-2015, doi:10.1002/hep.24315 (2011).
13 Moore, J. K. et al., Cytotherapy 17, 1604-1616, doi:10.1016/j.jcyt.2015.07.016 (2015).
14 Fraser, A. R. et al. Cytotherapy 19, 1113-1124, doi:10.1016/j.jcyt.2017.05.009 (2017).
15 Iredale, J. P. TInt J Biochem Cell Biol 29, 43-54, doi:10.1016/s1357-2725(96)00118-5 (1997).
16 Woo, C. H., Lim, J. H. & Kim, J. H. L J Immunol 173, 6973-6980, doi:10.4049/jimmunol.173.11.6973 (2004).
17 Lolmede, K. et al. I J Leukoc Biol 85, 779-787, doi:10.1189/jlb.0908579 (2009).
18 Liao, Y., Zhu, E. & Zhou, W. Ox-LDL Front Cardiovasc Med 8, 697236, doi:10.3389/fcvm.2021.697236 (2021).
19 Gratchev, A., Kzhyshkowska, J., Utikal, J. & Goerdt, S. Scand J Immunol 61, 10-17, doi:10.1111/j.0300-9475.2005.01524.x (2005).

20 Giannandrea, M. & Parks, W. C. Dis Model Mech 7, 193-203, doi:10.1242/dmm.012062 (2014).
21 Cabrera, S. et al. Int J Biochem Cell Biol 39, 2324-2338, doi:10.1016/j.biocel.2007.06.022 (2007).
22 Wang, Q. et al. Mol Med Rep 20, 5239-5248, doi:10.3892/mmr.2019.10740 (2019).
23 Campana, L. & Iredale, J. P. Semin Liver Dis 37, 1-10, doi:10.1055/s-0036-1597816 (2017).
24 Zhao, H. et al., World J Nephrol 2, 84-89, doi:10.5527/wjn.v2.i3.84 (2013).
25 Maretti-Mira, A et al. Acta Trop 119, 160-164, doi:10.1016/j.actatropica.2011.05.009 (2011).
26 Afratis, N. A., et al. Matrix Biol 68-69, 167-179, doi:10.1016/j.matbio.2018.02.007 (2018).
27 Iredale, J. P. Gastroenterology 126, 1199-1201, doi:10.1053/j.gastro.2004.01.060 (2004).
28 Rojkind, M., G et al Gastroenterology 76, 710-719 (1979).
29 Bearzi, C. et al. PCell Death Dis 5, e1053, doi:10.1038/cddis.2014.12 (2014).
30 Laurenzana, A. et al., Oncotarget 5, 3711-3727, doi:10.18632/oncotarget.1987 (2014)
31 van Wilgenburg B, Browne C, Vowles J, Cowley S. A, PloS One, 2013 doi.org/10.1371/journal.pone.0071098
32 Baghbaderani, B. A., et al. (2016). “Detailed Characterization of Human Induced Pluripotent Stem Cells Manufactured for Therapeutic Applications.” Stem Cell Rev Rep 12(4): 394-420.

1. A method for producing hypoimmunogenic functional macrophages, the method comprising:
(a) providing stem cells that are essentially devoid of functional HLA I and HLA II complexes on their surface; and
(b) differentiating the stem cells into hypoimmunogenic functional macrophages.

2. The method of claim 1, wherein the stem cells are pluripotent stem cells.

3. The method of claim 1 or 2, wherein the hypoimmunogenic functional macrophages display at least one or all of the following characteristics: capacity for phagocytosis, ability to polarise to an M1-like phenotype, and ability to polarise to an M2-like phenotype.

4. The method of claim 3, wherein capacity for phagocytosis is assessed by determining the percentage of cells phagocytosing in a representative sample of the hypoimmunogenic functional macrophages, and wherein optionally at least 60%, at least 70%, at least 80%, at least 90% or at least 95% of the cells are phagocytosing.

5. The method of any of claims 2-4, wherein the pluripotent stem cells are induced pluripotent stem cells (iPSCs).

6. The method of any of claims 2-5, wherein the pluripotent stem cells are isolated cells, a cell aggregate or an embryonic body.

7. The method of any of the preceding claims, wherein the stem cells are devoid of HLA I and II through knock-out or knock-down of at least one sequence encoding at least one unit in the HLA I and/or the HLA II complex, or a transcriptional activator thereof, optionally knock-out or knock-down of the B2M gene, the CIITA gene or a combination thereof.

8. The method of any of the preceding claims, wherein the stem cells comprise a genetic modification that prevents surface expression of HLA I and HLA II complexes.

9. The method of claim 8, wherein the genetic modification is transformation with a construct encoding an antisense oligonucleotide, siRNA, RNAi or miRNA targeting at least one unit in the HLA I and/or the HLA II complex or a transcriptional activator thereof, or wherein the genetic modification is mutation or deletion of sequence encoding at least one unit in the HLA I and/or the HLA II complex, or a transcriptional activator thereof.

10. The method of claim 8 or 9, wherein the genetic modification results in the expression of a silencing molecule from the genome of the stem cells targeting at least one unit in the HLA I and/or the HLA II complex, or a transcriptional activator thereof.

11. The method of any of claims 8-10, wherein the genetic modification comprises gene editing which results in knockout of at least one component of the HLA I and/or HLA II complexes or a transcriptional activator thereof, optionally wherein the gene editing results in knockout of B2M, CIITA or both.

12. The method of any one of claims 2-11, wherein the step of differentiating the pluripotent stem cells into hypoimmunogenic functional macrophages comprises a step of differentiating the pluripotent stem cells into embryonic bodies.

13. The method of claim 12, wherein the step of differentiating the pluripotent stem cells into embryonic bodies comprises incubating the pluripotent stem cells with at least one or all of BMP4, SCF and VEGF.

14. The method of claim 13, wherein the step of differentiating the pluripotent stem cells into embryonic bodies further comprises incubating the cells with Rock Inhibitor (Y-27632).

15. The method of any one of claims 2-14, wherein the method comprises a step of differentiating the embryonic bodies into macrophage progenitors.

16. The method of claim 15, wherein the step of differentiating the embryonic bodies into macrophage progenitors comprises culturing the embryonic bodies in the presence of M-CSF and/or IL-3.

17. The method of claim 16, wherein at least 80% of the macrophage progenitors produced by the step of differentiating the embryonic bodies into macrophage progenitors express at least one or all of CD45, CD14, CD206 and 25F9.

18. The method of any one of the preceding claims, wherein the method comprises a step of maturing the macrophage progenitors into functional macrophages.

19. The method of claim 18, wherein the step of maturing the macrophage progenitors into functional macrophages comprises culturing the macrophage progenitors in the presence of M-CSF.

20. The method of any one of the preceding claims, wherein the method further comprises polarising the macrophages by contacting the macrophages with IL-10.

21. A method of polarising macrophages derived from stem cells essentially devoid of functional HLA I and HLA II complexes on their surface to a pro-regenerative phenotype, the method comprising contacting the macrophages with IL-10.

22. The method of claim 20 or 21, wherein the contacting with IL-10 comprises providing IL-10 exogenously as a protein.

23. The method of claim 20 or 21 wherein the contacting with IL-10 comprises engineering the functional macrophage to express IL-10.

24. The method of claim 23, wherein the functional macrophage is engineered to express IL-10 transiently.

25. The method of claim 24, wherein the functional macrophage is engineered to express IL-10 transiently by transfecting the cell with DNA or RNA.

26. The method of claim 20 or 21, wherein contacting the macrophages with IL-10 comprises engineering the stem cell to express IL-10 stably.

27. The method of claim 26, wherein the stem cell is engineered to express IL-10 stably by introducing a sequence encoding IL-10 into the stem cell, optionally wherein the sequence encoding IL-10 is introduced into the nuclear genome of the stem cell.

28. The method of any preceding claims comprising formulating the hypoimmunogenic functional macrophages in a therapeutic composition.

29. A cell composition comprising macrophages obtained by the method of any of the preceding claims.

30. The cell composition of claim 29, for use in treating liver injury, optionally wherein the liver injury comprises liver fibrosis, inflammatory liver injury with a fibrotic element, chronic liver injury and/or liver cirrhosis.

31. The cell composition of claim 30, wherein the liver injury is liver cirrhosis.

32. A cell population comprising a pro-regenerative macrophage derived from a stem cell (SC), wherein said macrophage is genetically modified to knock down or knock out of expression of one or both of human leukocyte antigen class I (HLA I) and HLA class II (HLA-II), for use in treating a chronic inflammatory condition with a fibrotic element.

33. A cell population as claimed in claim 32 wherein the SC is an induced pluripotent stem cell (iPSC).

34. A cell population as claimed in claim 32 or claim 33 wherein said SC or cell derived therefrom is genetically modified to knock down or knock-out one or more genes or loci associated with HLA I and/or HLA II.

35. The cell population as claimed in claims 32-34, wherein the macrophage is genetically modified to knock down or knock out of expression of both HLA I and HLA II.

36. The cell population of claim 35, wherein the macrophage is genetically modified to knock out expression of both HLA I and HLA II.

37. The cell population of claim 36, wherein the macrophage is genetically modified to knock out endogenous expression of both HLA I and HLA II.

38. The cell population of claim 36 or 37, wherein genetically modifying the macrophage comprises knocking out both the beta-2 microglobulin (B2M) and the Class II Transactivator (CIITA) loci.

39. The cell population as claimed in any of claims 32-38, wherein the chronic inflammatory condition with a fibrotic element is liver fibrosis, optionally liver cirrhosis.

40. The cell population as claimed in any of claims 32-39, wherein the pro-regenerative macrophage has been polarised to the pro-regenerative phenotype by contacting the macrophage with IL-10.
